SEQUENCE LISTING (4 sequences)

SEQ ID NO: 1 - DNA, 17 bp, Artificial Sequence
  Forward Primer
accgagcgcggctacag

SEQ ID NO: 2 - DNA, 22 bp, Artificial Sequence
  Reverse Primer
cttaatgtcacgcacgatttcc

SEQ ID NO: 3 - DNA, 21 bp, Artificial Sequence
  Forward Primer
accgcacagatgcgtaaggag

SEQ ID NO: 4 - DNA, 21 bp, Artificial Sequence
  Reverse Primer
ttcattaatgcagctggcacg

(19) United States
(12) Patent Application Publication
(10) Pub. No.: US 2008/0268514 A1
(43) Pub. Date: Oct. 30, 2008

(54) SAMPLE STORAGE FOR LIFE SCIENCE

(76) Inventors: Rolf Muller, Del Mar, CA (US); Judy Muller-Cohn, Del Mar, CA (US)

Correspondence Address: SEED INTELLECTUAL PROPERTY LAW GROUP PLLC, 701 FIFTH AVE, SUITE 5400, SEATTLE, WA, 98104, US

(21) Appl. No.: 12/108,360

(22) Filed: Apr. 23, 2008

Related U.S. Application Data
(60) Provisional application No. 60/913,781, filed on Apr. 24, 2007.

(51) Int. Cl. C12N 11/00; C12N 5/06; C12N 5/04; C12N 1/20; C12N 1/14; C12N 7/00; C12N 9/00

(52) U.S. Cl. 435/174; 435/325; 435/410; 435/252.1; 435/255.1; 435/235.1; 435/183

(57) ABSTRACT

Compositions and methods are disclosed for liquid storage of biological samples with recovery of substantially all biological activity and without refrigeration. Also disclosed are compositions and methods for automated storing, tracking retrieving and analyzing of such liquid-storable biological samples, including nucleic acids and proteins (including enzymes). RFID-tagged liquid-storable biological sample storage devices featuring liquid matrices are disclosed, as also are computer-implemented systems and methods for managing sample data.

### CROSS-REFERENCE TO RELATED APPLICATION

This application claims the benefit under 35 U.S.C. § 119(e) of U.S. Provisional Patent Application No. 60/913,781 entitled “Sample Storage for Life Science” and filed on Apr. 24, 2007, which provisional application is incorporated herein by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 150079_{—}402_SEQUENCE_LISTING.txt. The text file is 1 KB, was created on Apr. 23, 2008, and is being submitted electronically via EFS-Web, concurrent with the filing of the specification.

### BACKGROUND OF THE INVENTION

1. Technical Field

The present invention relates generally to compositions and methods for biological sample storage. The invention also relates to the use, organization, storage, tracking, retrieval and analysis of such biological materials and samples and to the automation of these processes.

2. Description of the Related Art

Research in the life sciences field is based upon the analysis of biological materials and samples, such as DNA, RNA, blood, urine, buccal swabs, bacteria, archaebacteria, viruses, phage, plants, algae, yeast, microorganisms, PCR products, cloned DNA, proteins, enzymes, peptides, prions, eukaryotes (e.g., protoctisca, fungi, plantae and animalia), prokaryotes, cells and tissues, germ cells (e.g., sperm and oocytes), stem cells, and of minerals or chemicals. Such samples are typically collected or obtained from appropriate sources and placed into storage and inventory for further processing and analysis. Oftentimes, transportation of samples is required, and attention is given to preserve their integrity, sterility and stability. Biological samples can be transported in a refrigerated environment using ice, dry ice or other freezing facility. However, adequate low temperatures often cannot conveniently be maintained for extended time periods such as those required for transportation between countries or continents, particularly where an energy source for the refrigeration device is lacking.

Storage containers for such samples include bottles, tubes, vials, bags, boxes, racks, multi-well dishes and multi-well plates which are typically sealed by individual screw caps or snap caps, snap or seal closures, lids, adhesive strips or tape, or multi-cap strips. The standard container format for medium to high throughput of sample storage, processing and automation of biological processes is a 96-, 384-, or 1536-well plate or array. The containers and the samples contained therein are stored at various temperatures, for example at ambient temperature or at 4° C. or at temperatures below 0° C., typically at about −20° C. or at −70° C. to −80° C. The samples that are placed and stored in the devices are most frequently contained in liquid medium or a buffer solution, and they require storage at such subzero temperatures (e.g., −20° C. or −70 to −80° C.). In some cases, samples are first dried and then stored at ambient temperature (e.g., WO 2005/113147, US 2005/0276728, US 2006/0099567), or at 4° C., at −20° C. or at −70 to −80° C.

For example, presently, nucleic acids are stored in liquid form at low temperatures. For short term storage, nucleic acids can be stored at 4° C. For long-term storage the temperature is generally lowered to −20° C. to −70° C. to prevent degradation of the genetic material, particularly in the case of genomic DNA and RNA. Nucleic acids are also stored at room temperature on solid matrices such as cellulose membranes. Both storage systems are associated with disadvantages. Storage under low temperature requires costly equipment such as cold rooms, freezers, electric generator back-up systems; such equipment can be unreliable in cases of unexpected power outage or may be difficult to use in areas without a ready source of electricity or having unreliable electric systems. The storage of nucleic acids on cellulose fibers also results in a substantial loss of material during the rehydration process, since the nucleic acid remains trapped by, and hence associated with, the cellulose fibers instead of being quantitatively recoverable. Nucleic acid dry storage on cellulose also requires the subsequent separation of the cellulose from the biological material, since the cellulose fibers otherwise contaminate the biological samples. The separation of the nucleic acids from cellulose filters requires additional handling, including steps of pipetting, transferring of the samples into new tubes or containers, and centrifugation, all of which can result in reduced recovery yields and/or increased opportunity for the introduction of unwanted contaminants and/or exposure to conditions that promote sample degradation, and which are also cost- and labor-intensive.

Proteins are presently handled primarily in liquid form as solutions (e.g., in a compatible aqueous solution containing a salt and/or buffer) or suspensions (e.g., in a saturated ammonium sulfate slurry), in cooled or frozen environments typically ranging from −20° C. to storage in liquid nitrogen (Wang et al., 2007 J. Pharm. Sci. 96(1):1-26; Wang, 1999 Inter. J. of Pharm. 185: 129-188). In some exceptions proteins may be freeze-dried, or dried at room temperature in the presence of trehalose and applied directly to an untreated surface. (Garcia de Castro et al., 2000 Appl. Environ. Microbiol. 66:4142; Manzanera et al., 2002 Appl. Environ. Microbiol. 68:4328). Proteins often degrade and/or lose activity even when stored cooled (4° C.), or frozen (−20° C. or −80° C.). The freeze-thaw stress on proteins reduces bioactivity (e.g., enzymatic activity, specific binding to a cognate ligand, etc.) especially if repeated freeze-thawing of aliquots of a protein sample is required. The consequent loss of protein activity that may be needed for biological assays typically requires the readjustment of the protein concentration in order to obtain comparable assay results in successive assays, and oftentimes results in compromised reliability of experimental data generated from such samples.

Drying of proteins and nucleic acids has yet to be universally adopted by the research scientific, biomedical, biotechnology and other industrial business communities because of the lack of standard established and reliable processes, difficulties with recoveries of quantitative and functional properties, variable buffer and solvent compatibilities and tolerances, and other difficulties arising from the demands of handling nucleic acids and proteins. The same problems apply to the handling, storage, and use of other biological materials, such as viruses, phage, bacteria, cells and multicellular organisms. Dissacharides such as trehalose or lactitol, for example, have been described as additives for dry storage of protein-containing samples (e.g., U.S. Pat. No. 4,891,319; U.S. Pat. No. 5,834,254; U.S. Pat. No. 6,896,894; U.S. Pat. No. 5,876,992; U.S. Pat. No. 5,240,843; WO 90/05182; WO 91/14773), but usefulness of such compounds in the described contexts has been compromised by their serving as energy sources for undesirable microbial contaminants, by their limited stabilizing effects when used as described, by their lack of general applicability across a wide array of biological samples, and by other factors.

The highly labile nature of biological samples makes it extremely difficult to preserve their biological activity over extended time periods. While storing nucleic acids and proteins under freeze-dried conditions (e.g., as lyophilizates) can extend the storage life (shelf-life) of a sample, the subsequent loss of activity upon reconstitution in a liquid makes freeze-drying (e.g., lyophilization) a less than ideal storage technique. Moreover, drying methods cannot be used effectively for other biological materials such as those collected in large volumes, or as swabs of surfaces for biofilm collection, or for some viruses, bacteria, or multicellular organisms. For example, the ability to maintain liquid bacteria cultures under non-selective growth conditions would be particularly desirable during long term transportation, particularly in an environment that retards the growth rate and preserves the survival of the bacteria, but no such ability currently exists. Similarly, the ability to store samples stably and for extended periods in a liquid or semi-liquid environment at ambient or near-ambient temperatures (e.g., about 23° C. to 37° C.) thereby avoiding extreme temperatures (e.g., about 0° C. to −80° C.) would be highly advantageous in maintaining fully functional and intact biological samples, as these are native conditions for many biomolecules. Such capabilities are not, however, presently known.

The degradation of biological samples collected from distant places, be it a foreign country, continent, undersea or outer space, is also currently problematic, as proper analysis and testing of the samples are subsequently compromised and/or delayed. As such, presently available storage technologies for biological samples are not adequate, particularly with regard to preparation or collection of large quantities of proteins or other types of biomolecules that may not be amenable to dry storage, and/or to biological sample modalities for which it is desirable to have a storage capability for a time period of over one year or longer while retaining substantially constant biological activity. For example, in the case of disease outbreak or bioterrorism investigations, such an ability to preserve the integrity of biological samples could be needed, particularly if the sample is collected under extreme environmental conditions and then subjected to lengthy transportation to an appropriate facility for analysis. Thus, the ability to store biological samples for extended time periods without the need for time-consuming, impractical, inconvenient and/or costly preservation methods, particularly those that require refrigeration, would be highly advantageous.

Accordingly, there is clearly a need in the art for compositions and methods for storing biological samples in liquid form for extended time periods (e.g., in excess of one month, six months, nine months, one year, or longer) while maintaining the biological activity, for instance, for samples collected under extreme environmental conditions (e.g., conditions including, but not limited to, extreme temperatures (e.g., sub-zero or tropical), atmospheric conditions such as increased pressure (e.g., undersea) or low gravity (e.g., outer space), UV radiation, humidity, etc., particularly over extended time periods, without complicated preparations and storage conditions. The presently disclosed embodiments address these needs and offer other related advantages.

### BRIEF SUMMARY OF THE INVENTION

According to certain herein provided embodiments, there is provided a liquid-storable biological sample, comprising (a) a biological sample, (b) a liquid matrix that comprises a matrix material dissolved or dissociated in a biocompatible solvent and (c) at least one stabilizer, wherein (a), (b) and (c) are in fluid contact with one another for at least one day without refrigeration, and wherein substantially all biological activity of the liquid-storable biological sample is recoverable following storage without refrigeration for a time period of at least one day. In another embodiment, the liquid-storable biological sample comprises at least two stabilizers, where at least one stabilizer comprises a trehalase inhibitor, and wherein the matrix material comprises polyvinyl alcohol. In another embodiment, the liquid-storable biological sample comprises a glycosidase inhibitor that is selected from a trehalase inhibitor, a chitinase inhibitor, an α-glucosidase inhibitor, a glycogen phosphorylase inhibitor, a neuraminidase inhibitor, a ceramide glucosyltransferase inhibitor, and a lysosomal glycosidase inhibitor. In a further embodiment, the trehalase inhibitor is selected from suidatrestin, validamycin A, validoxylamine A, MDL 26537, trehazolin, salbostatin and casuarine-6-O-α-D-glucopyranoside.

In another embodiment, there is provided a liquid-storable biological sample wherein the matrix material is dissolved in the biocompatible solvent and wherein at least one stabilizer comprises an inhibitor that is a biological inhibitor or a biochemical inhibitor and the matrix material comprises polyvinyl alcohol, from about 0.1% to about 10% weight-to-volume polyvinyl alcohol, from about 0.5% to about 5% weight-to-volume polyvinyl alcohol, from about 1% to about 5% weight-to-volume polyvinyl alcohol, or from about 0.5% to about 1.5% weight-to-volume polyvinyl alcohol.

In certain further embodiments, there is provided a liquid-storable biological sample wherein the liquid matrix comprises a solution that is selected from a solution that comprises about 1% weight-to-volume polyvinyl alcohol, a solution that comprises about 3% weight-to-volume polyvinyl alcohol, a solution that comprises about 5% weight-to-volume polyvinyl alcohol, a solution that comprises about 1% weight-to-volume polyvinyl alcohol and about 5% weight-to-volume trehalose, a solution that comprises about 1% weight-to-volume polyvinyl alcohol and about 5% weight-to-volume validamycin, and a solution that comprises about 1% weight-to-volume polyvinyl alcohol, about 5% weight-to-volume trehalose and about 5% weight-to-volume validamycin.

In other embodiments, a liquid-storable biological sample is provided wherein the liquid matrix comprises a solution that is selected from a solution that comprises from about 1% weight-to-volume to about 5% weight-to-volume polyvinyl alcohol and about 5% weight-to-volume of a trehalase inhibitor, a solution that comprises about 1% weight-to-volume polyvinyl alcohol and about 1% to about 10% weight-to-volume of a trehalase inhibitor, and a solution that comprises about 1% weight-to-volume polyvinyl alcohol, about 5% weight-to-volume trehalose and about 5% weight-to-volume of a trehalase inhibitor. In certain further embodiments, the trehalase inhibitor is selected from suidatrestin, validamycin A, validoxylamine A, MDL 26537, trehazolin, salbostatin and casuarine-6-O-α-D-glucopyranoside.

In other embodiments, provided herein is a liquid-storable biological sample wherein the matrix material comprises at least one material selected from polyethylene glycol, agarose, poly-N-vinylacetamide, polyvinyl alcohol, carboxymethyl cellulose, 2-hydroxyethyl cellulose, poly(2-ethyl-2-oxazoline), poly(vinyl-pyrrolidone), poly(4-vinylpyridine), polyphenylene oxide, crosslinked acrylamide, polymethacrylate, carbon nanotubes, polylactide, lactide/glycolide copolymer, hydroxymethacrylate copolymer, calcium pectinate, hydroxypropyl methylcellulose acetate succinate, heparin sulfate proteoglycan, hyaluronic acid, glucuronic acid, thrombospondin-1 N-terminal heparin-binding domain, fibronectin, a peptide/water-soluble polymeric modifier conjugate and collagen. In certain further embodiments, the liquid-storable biological sample comprises a trehalase inhibitor that comprises validamycin.

In certain preferred embodiments, there is provided a liquid-storable biological sample wherein the biological sample comprises at least one of (i) an isolated biomolecule that is selected from DNA, RNA, a protein, a polypeptide, a lipid, a carbohydrate, glycoconjugate, an oligosaccharide, and a polysaccharide, (ii) a biological material that is selected from a mammalian cell, a non-mammalian cell, a plant cell, an animal cell, a bacterium, a microorganism, a yeast cell, a virus, a vaccine, blood, urine, a biological fluid, an environmental sample, and a buccal swab, and (iii) a bioactive small molecule.

According to certain here described embodiments, there is provided a liquid-storable biological sample, comprising: (a) a biological sample; (b) a liquid matrix that comprises polyvinyl alcohol dissolved in a biocompatible solvent; and (c) at least one stabilizer which comprises validamycin, wherein (a), (b) and (c) are in fluid contact with one another for at least one day without refrigeration, and wherein substantially all biological activity of the liquid-storable biological sample is recoverable following storage without refrigeration for a time period of at least one day.

In other embodiments, there is provided a liquid-storable biological sample comprising a buffer that is capable of maintaining a desired pH. In further embodiments, the buffer comprises a compound that is selected from Tris, citrate, acetate, phosphate, borate, HEPES, MES, MOPS, PIPES, carbonate and bicarbonate. In other embodiments, there is provided a liquid-storable biological sample wherein the biological inhibitor or biochemical inhibitor is selected from validamycin A, TL-3, sodium orthovanadate, sodium fluoride, N-α-tosyl-Phe-chloromethylketone, N-α-tosyl-Lys-chloromethylketone, aprotinin, phenylmethylsulfonyl fluoride and diisopropylfluoro-phosphate. In certain embodiments, the biological inhibitor or biochemical inhibitor is selected from a kinase inhibitor, a phosphatase inhibitor, a caspase inhibitor, a granzyme inhibitor, a cell adhesion inhibitor, a cell division inhibitor, a cell cycle inhibitor, a lipid signaling inhibitor and a protease inhibitor. In other certain embodiments, the biological inhibitor or biochemical inhibitor is selected from a reducing agent, an alkylating agent and an antimicrobial agent.

Turning to another embodiment, there is provided a liquid-storable biological sample which comprises at least one detectable indicator. In a further embodiment, the detectable indicator comprises a colorimetric indicator. In another embodiment, the detectable indicator comprises one or a plurality of GCMS tag compounds. In certain further embodiments, the detectable indicator is selected from a fluorescent indicator, a luminescent indicator, a phosphorescent indicator, a radiometric indicator, a dye, an enzyme, a substrate of an enzyme, an energy transfer molecule, and an affinity label. In yet other embodiments, the detectable indicator is selected from phenol red, ethidium bromide, a DNA polymerase, a restriction endonuclease, cobalt chloride, Reichardt's dye and a fluorogenic protease substrate. Also provided herein in certain embodiments is a liquid-storable biological sample wherein the detectable indicator is capable of detectably indicating presence of at least one of an amine, an alcohol, an aldehyde, a thiol, a sulfide, a nitrite, avidin, biotin, an immunoglobulin, an oligosaccharide, a nucleic acid, a polypeptide, an enzyme, a cytoskeletal protein, a reactive oxygen species, a metal ion, pH, Na⁺, K⁺, Cl⁻, a cyanide, a phosphate and selenium.

According to certain herein described embodiments, there is provided a liquid-storable biological sample wherein substantially all biological activity of the liquid-storable biological sample is recoverable following storage without refrigeration for a time period that is selected from (i) at least one week, (ii) at least one month, (iii) at least six months, (iv) at least nine months, (v) at least twelve months, (vi) at least eighteen months and (vii) at least twenty-four months.

Turning to another embodiment, there is provided herein a liquid-storable biological sample, comprising: (a) a biological sample; (b) a liquid matrix that comprises a matrix material dissolved or dissociated in a biocompatible solvent; and (c) at least one stabilizer, wherein (a), (b) and (c) are in fluid contact with one another for at least one day without refrigeration, and wherein substantially all biological activity of the liquid-storable biological sample is recoverable following storage without refrigeration for a time period of at least one day, wherein: (I) the matrix material of (b) does not covalently self-assemble and has the structure:

—[—X—]ₙ—

wherein X is —CH₃, —CH₂—, —CH₂CH(OH)—, substituted —CH₂CH(OH)—, —CH₂CH(COOH)—, substituted —CH₂CH(COOH)—, —CH═CH₂, —CH═CH—, C₁-C₂₄ alkyl or substituted alkyl, C₂₋₂₄ alkenyl or substituted alkenyl, polyoxyethylene, polyoxypropylene, or a random or block copolymer thereof; and wherein n is an integer having a value of about 1-100, 101-500, 501-1000, 1001-1500, or 1501-3000; and wherein (II) the stabilizer is not covalently linked to the polymer and comprises trehalose, a trehalase inhibitor, or a compound that is selected from D-(+)-raffinose, β-gentiobiose, ectoine, D-(+)-raffinose pentahydrate, myo-inositol, hydroxyectoine, magnesium D-gluconate, 2-keto-D-gluconic acid hemicalcium salt hydrate, D(+)-melezitose, calcium lactobionate monohydrate, β-lactose, turanose, and D-maltose.

In certain further embodiments, the polymer is capable of non-covalent association with at least one stabilizer. In certain other further embodiments, the polymer is capable of non-covalent association with at least one of a nucleic acid molecule and a polypeptide.

In other embodiments, there is provided herein a method of storing a biological sample, comprising: (a) contacting a biological sample and a liquid matrix, the liquid matrix comprising (i) a matrix material dissolved or dissociated in a biocompatible solvent and (ii) at least one stabilizer, to obtain a liquid-storable biological sample; and (b) maintaining the liquid-storable biological sample for a time period of at least one day without refrigeration, wherein substantially all biological activity of the liquid-storable biological sample is recoverable following storage without refrigeration for the time period of at least one day. In further embodiments, methods are provided wherein following storage without refrigeration for said time period, degradation of the biological sample is decreased relative to degradation of a control biological sample maintained in the biocompatible solvent without refrigeration for the time period in the absence of the matrix material. In a further preferred embodiment, provided are methods wherein following storage without refrigeration for said time period, degradation of the biological sample is decreased relative to degradation of a control biological sample maintained in the biocompatible solvent without refrigeration for the time period in the absence of at least one of the matrix material and the stabilizer.

In certain embodiments, methods are provided wherein the step of contacting comprises simultaneously dissolving or dissociating the matrix material in the solvent, or the step of contacting is preceded by dissolving or dissociating the matrix material in the solvent, or the step of contacting is followed by dissolving or dissociating the matrix material in the solvent.

According to certain embodiments described herein are a method of preparing a liquid-storable biological sample storage device for one or a plurality of liquid-storable biological samples, comprising: (a) administering a liquid matrix to one or a plurality of sample wells of a biological sample storage device, wherein (1) said biological sample storage device comprises (i) a lid, and (ii) a sample plate comprising one or a plurality of sample wells that are capable of containing a biological sample, and wherein (2) the liquid matrix comprises (i) a matrix material that is dissolved or dissociated in a biocompatible solvent; and (ii) at least one stabilizer; (b) simultaneously or sequentially with step (a) and in either order, administering a biological sample to one or more of the sample wells; and (c) maintaining the biological sample storage device containing the liquid matrix and the biological sample without refrigeration for a time period of at least one day subsequent to step (b), wherein substantially all biological activity of the liquid-storable biological sample is recoverable following said time period, and thereby preparing the liquid-storable biological sample storage device.

In other embodiments, methods are provided wherein the step of administering comprises administering a liquid solution or a liquid suspension that contains the matrix material and the solvent. In other embodiments, there is provided a method wherein at least one well comprises at least one detectable indicator, where the indicator comprises a calorimetric indicator, or wherein the indicator comprises one or a plurality of GCMS tag compounds.

In certain preferred embodiments, methods are provided wherein the detectable indicator is selected from a fluorescent indicator, a luminescent indicator, a phosphorescent indicator, a radiometric indicator, a dye, an enzyme, a substrate of an enzyme, an energy transfer molecule, and an affinity label. In other embodiments, there are provided methods wherein the detectable indicator is capable of detectably indicating the presence of at least one of an amine, an alcohol, an aldehyde, a thiol, a sulfide, a nitrite, avidin, biotin, an immunoglobulin, an oligosaccharide, a nucleic acid, a polypeptide, an enzyme, a cytoskeletal protein, a reactive oxygen species, a metal ion, pH, Na⁺, K⁺, Cl⁻, a cyanide, a phosphate and selenium. In further embodiments, there is provided a method wherein the detectable indicator is selected from phenol red, ethidium bromide, a DNA polymerase, a restriction endonuclease, cobalt chloride, Reichardt's dye and a fluorogenic protease substrate. In certain further embodiments there are provided methods wherein at least one well comprises at least one stabilizer that is a biological inhibitor or a biochemical inhibitor.

In another embodiment, there is provided a method of recovering a stored biological sample, comprising: (a) contacting, simultaneously or sequentially and in either order in a biological sample storage device, one or a plurality of biological samples with a liquid matrix for storage of a biological sample, wherein (1) said biological sample storage device comprises (i) a lid, and (ii) a sample plate comprising one or a plurality of sample wells that are capable of containing the biological sample, and wherein (2) the matrix comprises (i) a matrix material that is dissolved or dissociated in a biocompatible solvent, and (ii) at least one stabilizer, to obtain one or a plurality of liquid-storable biological samples; (b) maintaining the biological sample storage device without refrigeration for a time period of at least one day subsequent to the step of contacting; and (c) removing the one or a plurality of liquid-storable biological samples from the biological sample storage device, wherein substantially all biological activity of the liquid-storable biological samples is recoverable following storage without refrigeration for the time period of at least one day, and thereby recovering said stored biological samples. In a certain preferred embodiment, there is provided a method wherein biological activity of the sample subsequent to the step of maintaining is substantially the same as biological activity of the sample prior to the step of contacting. In certain further embodiments there is provided a method wherein the biocompatible solvent is an activity buffer.

In another embodiment, there is provided herein a liquid-storable biological sample, comprising: (a) a biological sample; (b) a liquid matrix that comprises a matrix material dissolved or dissociated in a biocompatible solvent; (c) at least one stabilizer; and (d) an activity buffer, wherein (a), (b), (c) and (d) are in fluid contact with one another for at least one day without refrigeration, and wherein substantially all biological activity of the liquid-storable biological sample is recoverable following storage without refrigeration for a time period of at least one day. In certain further embodiments the activity buffer comprises a composition that is selected from the group consisting of a pH buffer, a free radical trapping agent, and a pathogen-neutralizing agent.

In another embodiment, there is provided a method of storing a biological sample, comprising: (a) contacting a biological sample and a liquid matrix to obtain a liquid-storable biological sample, the liquid matrix comprising a matrix material dissolved or dissociated in a biocompatible solvent; and (b) maintaining the liquid-storable biological sample for a time period of at least one day without refrigeration, wherein substantially all biological activity of the liquid-storable biological sample is recoverable following storage without refrigeration for the time period of at least one day. In certain preferred embodiments, there is provided a method wherein degradation of the biological sample is decreased relative to degradation of a control biological sample maintained in the biocompatible solvent without refrigeration in the absence of the matrix material.

In a further embodiment, there is provided a method wherein the step of contacting comprises simultaneously dissolving or dissociating the matrix material in the solvent, or wherein the step of contacting is preceded by dissolving or dissociating the matrix material in the solvent, or wherein the step of contacting is followed by dissolving or dissociating the matrix material in the solvent.

In another embodiment, there is provided a method of preparing a liquid-storable biological sample storage device for one or a plurality of liquid-storable biological samples, comprising: (a) administering a liquid matrix to one or a plurality of sample wells of a biological sample storage device, wherein (1) said biological sample storage device comprises (i) a lid, and (ii) a sample plate comprising one or a plurality of sample wells that are capable of containing a biological sample, and wherein (2) the matrix comprises a matrix material that is dissolved or dissociated in a biocompatible solvent; and (b) simultaneously or sequentially with step (a) and in either order, administering a biological sample to one or more of the sample wells; and (c) maintaining the biological sample storage device containing the liquid matrix and the biological sample without refrigeration for a time period of at least one day subsequent to step (b), wherein substantially all biological activity of the liquid-storable biological sample is recoverable following said time period, and thereby preparing the liquid-storable biological sample storage device. Further provided is a method of wherein the step of administering comprises administering a liquid solution that contains the matrix material and the solvent.

Turning to another embodiment there is provided a method of recovering a stored biological sample, comprising: (a) contacting, simultaneously or sequentially and in either order in a biological sample storage device, one or a plurality of biological samples with a liquid matrix for storage of a biological sample, wherein (1) said biological sample storage device comprises (i) a lid, and (ii) a sample plate comprising one or a plurality of sample wells that are capable of containing the biological sample, and wherein (2) the matrix comprises a matrix material that is dissolved or dissociated in a biocompatible solvent, to obtain one or a plurality of liquid-storable biological samples; (b) maintaining the biological sample storage device containing the liquid matrix and the biological sample without refrigeration for a time period of at least one day subsequent to the step of contacting; and (c) removing the one or a plurality of liquid-storable biological samples from the biological sample storage device, wherein substantially all biological activity of the liquid-storable biological samples is recoverable following storage without refrigeration for the time period of at least one day, and thereby recovering said stored biological samples. In certain embodiments, there is provided a method wherein the biological activity of the sample subsequent to the step of maintaining is substantially the same as biological activity of the sample prior to the step of contacting, wherein the biocompatible solvent comprises an activity buffer, and wherein the matrix material comprises polyvinyl alcohol.

According to certain embodiments described herein, there is provided a liquid-storable biological sample, comprising: (a) a biological sample; (b) a liquid matrix that comprises a matrix material dissolved or dissociated in a biocompatible solvent; and (c) a sample treatment composition, wherein (a), (b) and (c) are in fluid contact with one another for at least one day without refrigeration, and wherein substantially all of the liquid-storable biological sample is recoverable following storage without refrigeration for a time period of at least one day.

In further embodiments, there is provided a liquid-storable biological wherein the sample treatment composition comprises a composition that is selected from an activity buffer, a cell lysis buffer, a free radical trapping agent, a sample denaturant and a pathogen-neutralizing agent. In certain further embodiments, the liquid-storable biological sample is formulated to be isotonic, hypertonic or hypotonic.

In another embodiment, there is provided a method of identifying a stabilizer of a biological sample, comprising: (a) storing, for at least one day without refrigeration, a biological sample in a liquid matrix which comprises a matrix material that is dissolved or dissociated in a biocompatible solvent in the presence of a candidate agent; (b) recovering the biological sample; (c) comparing biological activity of the biological sample to the biological activity of a control sample that is stored for at least one day without refrigeration in the liquid matrix in the absence of the candidate agent, wherein retention of substantially all of the biological activity by the biological sample that is stored in the presence of the candidate agent and loss of biological activity by the control sample that is stored in the absence of the candidate agent indicates the candidate agent is a biological inhibitor or biochemical inhibitor, and thereby identifying a stabilizer of the biological sample. In certain further embodiments, the stabilizer is a biological inhibitor or a biochemical inhibitor.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE SEVERAL VIEWS OF THE DRAWINGS

FIG. 1 shows integrity of genomic DNA extracted from whole blood stored in liquid storage matrix at room temperature. (FIG. 1, left bar) QPCR analysis was used to measure the integrity of genomic DNA extracted from whole blood stored in 1% PVA basic liquid storage matrix for 4 months at ambient temperature. As a control, whole blood was stored at −20° C. without liquid storage matrix (FIG. 1, right bar). The integrity of genomic DNA was maintained when whole blood was stored in liquid storage matrix at room temperature, while it was significantly diminished in samples derived from whole blood stored frozen.

FIG. 2 shows storage of RNA at room temperature in liquid storage matrix prevented degradation. A 0.8% agarose gel stained with ethidium bromide is shown following separation of RNA fragments after storage in 1% PVA basic liquid storage matrix at room temperature for six days (SM, Lane 3). Essentially no significant degradation was detected as compared to identical samples that were stored frozen (−20° C., Lane 1). RNA was also stored in water at room temperature (noSM, Lane 2). RNA stored in water was significantly denatured compared to samples stored in 1% PVA based liquid matrix and maintained at either at room temperature or −20° C. for 6 days.

FIG. 3 shows that plasmid DNA (PDNA) stored in liquid storage matrix without refrigeration remained intact. A sample of 1 ng pUC19 was stored in 1% PVA basic liquid storage matrix or water. Samples were heated at 70° C. for 3 days. Control PDNA was stored in water at −20° C. (C lanes). The samples were then used as templates in PCR amplification reactions and 10 μl of each amplified product was run on an 0.8% agarose gel and then stained with ethidium bromide for analysis. Plasmid DNA stored in liquid storage matrix (SM lanes) showed robust amplification comparable to the frozen control (C lanes), while DNA stored in water could not be amplified (H₂O lanes). NC: no template control (NC lanes).

FIG. 4 shows that Taq polymerase stored in liquid storage matrix without refrigeration retained enzymatic activity. Taq polymerase (2.5 U) was stored in liquid storage matrix (SM) for 21 days at either room temperature (25° C.) or 50° C. An identical sample was also stored in water. Taq polymerase was stored at −20° C. (C) as a positive control. Aliquots of the stored enzymes were then used in PCR reactions with 50 ng pUC19 as template. A 10 μl sample of each reaction product was run on a 0.8% agarose gel that was then stained with ethidium bromide. Taq polymerase stored in liquid storage matrix for 21 days at either 25° C. (Lanes 1-4) or 50° C. (Lanes 5-6) showed robust PCR amplification. Amplification of template was comparable to the frozen control (C: Lane C). Taq polymerase stored in water only failed to amplify (H₂O: Lane H₂O). NC: no template control (NC: Lane NC). These results indicate that enzymatic function was retained when the polymerase was stored in liquid storage matrix, even after 21 days at elevated temperatures.

FIG. 5 shows that E. coli stored in liquid storage matrix at room temperature remained viable and plasmid DNA remained intact. E. coli cells containing pFIV-C plasmid were stored for 2 months at room temperature in liquid storage matrix (SM) or liquid Luria Broth (LB). Aliquots of stored samples were then used to grow overnight cultures before extraction of plasmid DNA that was then digested with EcoRI. Digested DNA samples were run on an 0.8% agarose gel and stained with ethidium bromide. Control DNA was used as a reference for integrity of the plasmid (+: Lane 3). E. coli stored in liquid storage matrix at room temperature retained plasmid DNA that could be digested with restriction enzymes (SM: Lane 1), while bacteria stored in LB no longer harbored the plasmid (LB: Lane 2).

FIG. 6 is a schematic diagram of a known radio-frequency communication system.

FIG. 7 is a schematic diagram of a system formed in accordance with one embodiment of the present invention.

FIG. 8 is a block diagram of a computer-implemented system architecture formed in accordance with another aspect of the present invention.

FIG. 9 shows a computer-implemented system architecture in accordance with certain invention embodiments.

FIG. 10 shows a computer-implemented system architecture in accordance with certain embodiments.

### DETAILED DESCRIPTION

The present invention is directed in certain embodiments as described herein to compositions and methods for substantially liquid storage of a biological sample, based on the unexpected discovery that in the presence of certain matrix materials and, in certain further embodiments, one or more stabilizers, a biological sample can be stored in liquid or semi-liquid form at ambient temperature for extended periods of time, such that substantially all of the biological activity of the sample can be recovered. As described herein, certain embodiments relate in part to advantages provided by selection of matrix materials which are compatible with preserving structure and/or activity of a biological sample, and in part to surprising advantages provided by selection of a stabilizer such as a trehalase inhibitor having antimicrobial activity, for use in long-term ambient temperature liquid-phase storage.

These and related embodiments permit efficient, convenient and economical storage of a wide variety of biological samples including, but not limited to, polynucleotides, enzymes and other proteins, and cells, without refrigeration or frozen storage. Samples may be stored in the liquid matrix at ambient temperature and following storage, the samples may be used immediately without a need for separating the sample from the matrix material, which does not interfere with biological activity of the sample. Embodiments provided herein offer advantageously superior recoveries of stored biological samples, including enhanced detection sensitivity for interrogating samples containing minute quantities of biomolecules of interest, and may find uses in clinical, healthcare and diagnostic contexts, in biomedical research, biological research and forensic science, and in biological products and other settings where sample storage and management for life sciences may be desired.

The invention may be used for storage of liquid or non-liquid samples and for storage at ambient temperature, and also may have use for the storage of diverse biological materials and biological samples, such as but not limited to DNA, RNA, blood, urine, other biological fluids (e.g., serum, serosal fluids, plasma, lymph, cerebrospinal fluid, saliva, mucosal secretions of the secretory tissues and organs, vaginal secretions, ascites fluids, fluids of the pleural, pericardial, peritoneal, abdominal and other body cavities, cell and organ culture medium including cell or organ conditioned medium, lavage fluids and the like, etc.) buccal swabs, bacteria, viruses, engineered viral vectors, yeast cells, vaccines (e.g., natural or synthetic, live or attenuated in the case of intact biological particles such as viral or other microbial vaccines, or extracts of natural, synthetic or artificial materials including products of genetic engineering), cells and tissues, cell or tissue lysates, cell or tissue homogenates or extracts, and the like, or other biological samples.

Biological samples may therefore also include a blood sample, biopsy specimen, tissue explant, organ culture, biological fluid or any other tissue or cell preparation, or fraction or derivative thereof or isolated therefrom, from a subject or a biological source. The subject or biological source may be a human or non-human animal, including mammals and non-mammals, vertebrates and invertebrates, and may also be any other multicellular organism or single-celled organism such as a eukaryotic (including plants and algae) or prokaryotic organism or archaeon, microorganisms (e.g., bacteria, archaea, fungi, protists, viruses), aquatic plankton, soil, biofilms, microbial mats or clusters, a primary cell culture or culture adapted cell line including but not limited to genetically engineered cell lines that may contain chromosomally integrated or episomal recombinant nucleic acid sequences or artificial chromosomes, immortalized or immortalizable cell lines, somatic cell hybrid cell lines, differentiated or differentiable cell lines, stem cells, germ cells (e.g., sperm, oocytes), transformed cell lines and the like.

The presently used terms “biological sample”, “biological molecule” and “biomolecule” encompass any substances and compounds substantially of biological origin that have properties that are relevant within the framework of scientific, diagnostic and/or pharmaceutical applications. Encompassed are not only native molecules, such as those that can be isolated from natural sources, but also forms, fragments and derivatives derived therefrom, as well as recombinant forms and artificial molecules, as long as at least one property of the native molecules is present. Preferred biological samples are those that can be applied for analytical, diagnostic and/or pharmaceutical purposes, such as, but not limited to, nucleic acids and their derivatives (e.g., oligonucleotides, DNA, cDNA, PCR products, genomic DNA, plasmids, chromosomes, artificial chromosomes, gene transfer vectors, RNA, mRNA, tRNA, siRNA, miRNA, hnRNA, ribozymes, peptide nucleic acid (PNA)), polypeptides and proteins (e.g., enzymes, receptor proteins, protein complexes, peptide hormones, antibodies, lipoproteins, glycoproteins, inteins, prions), as well as biologically active fragments thereof, carbohydrates and their derivatives (e.g., glycolipids, glycosylated proteins, glycosides, oligosaccharides, mono- and poly-saccharides, and glycosaminoglycans), and lipids and their derivatives (e.g., fats, fatty acids, glycerides, triglycerides, phospholipids, steroids, prostaglandins, and leukotrienes).

It will be clear to one of skill in the art, based on the present disclosure, that the compositions and processes according to embodiments encompassed by the present invention can also be applied to cellular tissues and to complete cells, as well as to portions thereof (e.g., organelles, membranes and membrane fragments, homogenates, extracts, subcellular fractions, lysates, etc.) as long as such derived portions are carriers of the above described biomolecules. For this reason, tissues, cells and portions thereof and the like are basically encompassed by the term “biological sample”.

Accordingly, the term “biological sample” may be regarded in its broadest sense, for instance, to refer to a vertebrate or invertebrate cell or tissue, for example in the case of vertebrate cells, to a fish cell (e.g., a zebrafish cell, or a pufferfish cell, etc.), an amphibian cell (e.g., a frog cell), an avian cell, a reptilian cell, a mammalian cell, etc. Examples of mammals include humans or non-human mammals, such as a monkey, ape, cow, sheep, goat, buffalo, antelope, oxen, horse, donkey, mule, deer, elk, caribou, water buffalo, camel, llama, alpaca, rabbit, pig, mouse, rat, guinea pig, hamster, dog, cat, etc. Also envisaged in other embodiments are biological samples that may comprise non-mammalian animals or organs derived therefrom, including, for example, annelids, mollusks, sponges, cnidaria, arthropods, amphibians, fish, birds and reptiles.

In certain other embodiments a biological sample may refer to microorganisms that are derived from aquatic plankton, animal tissues and organs as described above, microbial mats, clusters, sludge, flocs, or biofilms. Microorganisms of the “aquatic” plankton comprises bacterial plankton, archael plankton, viruses and phytoplankton, as well as zooplankton.

According to other embodiments, the term “biological sample” may include a specimen or culture obtained from any source (animal, plant, bacteria, virus, etc.) such as a subject or biological source, as well as from biological and environmental samples. Biological samples may be obtained from any vertebrate or non-vertebrate and may encompass fluids, solids, tissues, and gases. Environmental samples include environmental material such as surface matter, soil, water, biofilms, microbial mats, industrial samples, etc.

As used herein, “soil” is the complex product of geological and biological processes acting on inorganic minerals and biomass deposited on the earth's surface. It contains the majority of biodiversity on earth (Whitman et al., (1998) Proc. Natl. Acad. Sci. USA, 95(12, 6578-83) acting to recycle and biomineralize organic matter, and serves as a substratum to anchor and nourish higher plants.

Biofilms are microbial assemblages on the surface in “aqueous environments” in which microbes are embedded in a hydrated polymeric matrix. This matrix acts like a glue, holding the microbes together, attaching them to the surface and protecting them from detrimental external influences. They may contain several taxonomically distinct species (e.g., bacteria, fungi, algae, and protozoa), and may form on solid or liquid surfaces of diverse composition, such as metals, glass, plastics, tissue, minerals, and soil particles. Microbial mats and cluster are microbial assemblage/aggregates similar to biofilms in composition, however, not necessarily as firmly attached as solid surfaces.

Certain embodiments relate to a biological sample that may comprise an isolated biomolecule, where the term “isolated” means that the material is removed from its original environment (e.g., the natural environment if it is naturally occurring). For example, a naturally occurring nucleic acid or polypeptide present in an intact cell or in a living animal is not isolated, but the same nucleic acid or polypeptide, separated from some or all of the co-existing materials in the natural system, is isolated. Such nucleic acids could be part of a vector and/or such nucleic acids or polypeptides could be part of a composition, and still be isolated in that such vector or composition is not part of its natural environment.

Certain herein described embodiments relate to stabilization and/or preservation of a biological sample, which involves maintenance, retention or reconstitution of the structural and/or functional integrity of biological samples (including of molecular, multimolecular or oligomeric, organellar, subcellular, cellular, multicellular, or higher organizational levels of biological structure and/or function) and of the biological properties based thereupon. The biological activity of a biological sample that comprises, in a particular embodiment, a macromolecule or biopolymer or the like such as a polypeptide or polynucleotide, may involve, for example, the extensive maintenance of its primary, secondary and/or tertiary structure. The biological activity of a nucleic acid probe comprises, for example, its property of forming in a sequence-specific manner a hybridization complex (e.g., a duplex) with a nucleic acid target which is complementary to the probe. The biological activity of an antibody comprises, for example, a specific binding interaction with its cognate antigen.

As described herein, the biological activity of a substance means any activity which can affect any physical or biochemical properties of a biological system, pathway, molecule, or interaction relating to an organism, including for example but not limited to, viruses, bacteria, bacteriophage, prions, insects, fungi, plants, animals, and humans. Examples of substances with biological activity include, but are not limited to, polynucleotides, peptides, proteins, enzymes, antibodies, small molecules (e.g., a bioactive small molecule), pharmaceutical compositions (e.g., drugs), vaccines, carbohydrates, lipids, steroids, hormones, chemokines, growth factors, cytokines, liposomes, and toxins, liposomes. Persons familiar with the relevant art will recognize appropriate assays and methods for determining the biological activity of substances that affect the physical or biochemical properties of a biological system, for example, one or more biological activities that may include, but are not limited to, gene expression (see, e.g., Asubel, F M et al. (Eds.). 2007. Current Protocols in Molecular Biology, Wiley and Sons, Inc. Hoboken, N.J.), receptor-ligand interactions (see for example, Coligan et al. (Eds.). 2007. Current Protocols in Immunology, Wiley and Sons, Inc. Hoboken, N.J.), enzymatic activity (see, e.g., Eisenthal and Hanson (Eds.), Enzyme Assays, Second Edition. Practical Approaches series, No. 257.2002, Oxford University Press, Oxford, UK; Kaplan and Colowick (Eds.), Preparation and Assay of Enzymes, Methods in Enzymology, (vols. 1, 2 and 6). 1955 and 1961, Academic Press, Ltd., Oxford, UK), cytokine, hormone and bioactive peptide activities and other cell proliferation (e.g., mitogenic) and/or differentiation activities (see for example, Coligan et al. (Eds.). 2007 Current Protocols in Immunology, Wiley and Sons, Inc. Hoboken, N.J.), signal transduction (see for example, Bonifacino et al. (Eds.) 2007 Current Protocols in Cell Biology, Wiley and Sons, Inc. Hoboken, N.J.) and cell toxicity (e.g., cytotoxicity, excitotoxicity) (see for example, Bus J S et al. (Eds) 2007 Current Protocols in Toxicology, Wiley and Sons, Inc. Hoboken, N.J.), apoptosis and necrosis (Green and Reed, 1998 Science 281(5381):1309-12; Green D R, 1998 Nature Dec 17: 629; Green D R, 1998 Cell 94(6):695-69; Reed, J C (Ed.), 2000 Apoptosis, Methods in Enzymology (vol. 322), Academic Press Ltd., Oxford, UK).

In certain embodiments, the invention relates to the long-term storage of biological, chemical and biochemical material under substantially liquid conditions, and in a manner ready for immediate use. As described herein, there are provided embodiments which include a) the liquid storage matrix, b) preparation and optimization of the liquid sample matrix with compositions that increase the durability of the long-term storage conditions, including in certain embodiments, e.g., the use of a stabilizer which may be a biological or biochemical inhibitor, for instance a stabilizer such as a trehalase inhibitor having antimicrobial activity, and c) the process of simplifying complex biochemical processes of biologically active materials through the use of the liquid storage matrix.

These and related embodiments thus provide advantages associated with liquid or semi-liquid storage of biological samples stored without refrigeration, including improved stabilization and preservation of biological activity in biological samples, reduced degradation of biological samples during storage at room temperature in liquid or semi-liquid form (e.g., hydrogel) (and in particular through the use of a protective matrix), and simplification of the processes for preparing biological samples for further use by reducing or eliminating the need for time-consuming re-calibration and aliquoting of such samples, and by eliminating the need for physically separating a sample from the storage medium. Embodiments as described herein additionally provide superior biological sample recoveries by reducing or eliminating factors that can otherwise reduce sample recovery yields, such as undesirable sample denaturation and/or sample loss due to adsorption of the sample on sample container surfaces.

As used herein, “hydrogel” is not to be considered as limited to gels which contain water, but extend generally to all hydrophilic gels and gel composites, including those containing organic non-polymeric components in the absence of water. A gel is a state of matter that is intermediate between solids and liquids, and which consists of a solvent inside a three dimensional network.

According to certain embodiments the invention allows for purification and size fractionation of DNA, RNA, proteins and other biomolecules, cells, cellular components and other biological materials, minerals, chemicals, or compositions derived from a biological sample or other life sciences related sample. In certain embodiments the invention thus readily permits, for example, the use of one or a plurality of biological materials and/or biological samples in the performance of molecular biology procedures, including but not limited to polymerase chain reaction or PCR (including RT-PCR), biopolymer (e.g., polynucleotide, polypeptide, oligosaccharide or other biopolymer) sequencing, oligonucleotide primer extension, haplotyping (e.g., DNA haplotyping) and restriction mapping in one unified, integrated and easy-to-use platform. The invention also readily permits, for example and in certain embodiments, the use of one or a plurality of biological samples and/or biological materials for the performance of protein crystallography. In other embodiments there is provided a platform for use, testing or detection (including diagnostic applications) of an antibody or small molecule (whether naturally occurring or artificial, such as a bioactive small molecule) or other biological molecule (e.g., a “biomolecule”), for example, a protein, polypeptide, peptide, amino acid, or derivative thereof; a lipid, fatty acid or the like, or derivative thereof; a carbohydrate, saccharide or the like or derivative thereof, a nucleic acid, nucleotide, nucleoside, purine, pyrimidine or related molecule, or derivative thereof, or the like; or another biological molecule that is a constituent of a biological sample.

### Liquid Storage of a Biological Sample

Compositions and methods described herein relate to liquid and/or substantially liquid storage of a biological sample, and may include the use of any suitable container, including, for example, a liquid storage device. The liquid storage device is an application of the biological sample storage device as herein disclosed, which contains a matrix material for use as a liquid or substantially liquid (e.g., hydrogel) sample matrix, including in certain preferred embodiments a matrix material that dissolves or dissociates in a solvent as described herein, for long-term storage of a biological sample or a biological material, such as but not limited to blood, urine and other biological fluids, bacteria, parasites, cells, tissues, viruses and viral vectors, chemical compounds (whether naturally occurring or artificially produced), plasmid DNA, DNA fragments, oligonucleotides, peptides, fluorogenic substrates, genomic DNA, PCR products, cloned DNA, artificial chromosome, RNA, proteins, enzymes, polypeptides, prions, vaccines, plants and algae, minerals and chemicals, and other biological samples as disclosed herein.

These and related embodiments derive from the observation that stable, long-term liquid storage of biological samples or biological materials may be effected without refrigeration when such samples or materials are stored in a matrix material such as those described herein, including a liquid matrix material. According to non-limiting theory, biological materials present in a biological sample may interact with the matrix material by specific or non-specific binding or other mechanism of attachment, including those involving formation of non-covalent and/or covalent chemical bonds and or intermolecular associative interactions such as hydrophobic and/or hydrophilic interactions, hydrogen bond formation, electrostatic interactions, and the like. Accordingly, the present invention provides devices for stable, long-term liquid or semi-liquid storage of biological samples at common indoor ambient room temperatures (e.g., typically 20-27° C. but varying as a function of geography, season and physical plant from about 15-19° C. or about 18-23° C. to about 22-29° C. or about 28-32° C.) for use in the sample data processing methods and systems described herein.

Preferred embodiments involve the use of sample storage devices as described herein that comprise a liquid matrix material which is capable of liquid or semi-liquid storage of a biological sample or a biological material without refrigeration, for example, at ambient room temperature. In certain preferred embodiments, there is little or no evaporation of biocompatible solvent (e.g., water) that is allowed to transpire by conditions under which the liquid-storable biological sample is maintained. The samples are preferably stored under liquid or substantially liquid conditions that stabilize the sample, i.e., little or no detectable (e.g., with statistical significance) degradation or undesirable chemical or physical modification of the sample occurs, according to criteria that will vary as a factor of the nature of the sample being stored and that will in any event be familiar to those having skill in the relevant art. As such, it will be appreciated from the present disclosure that according to certain preferred embodiments one or more of the sample, matrix material and stabilizer will be in fluid contact with one another, e.g., present within a common liquid phase, such as a biocompatible solvent.

Non-limiting examples of sample storage devices may include, bottles, tubes, vials, bags, boxes, racks, multi-well dishes and multi-well plates, which are typically sealed by individual screw caps or snap caps, snap or seal closures, lids, adhesive strips or tape, or multi-cap strips. Other containers and vessels suitable for liquid-storable biological samples as described herein will be known to those familiar with the art, such as for example, specimen collection containers. In certain embodiments, the standard container format for medium to high throughput of sample storage, processing and automation of biological processes is a 96-, 384-, or 1536-well plate or array. Other information regarding biological sample storage devices in general may be found, for example, in US/20050276728 and US/20060099567, including references cited therein.

Certain preferred embodiments provide compositions and methods for storing biological material (e.g., genomic DNA, plasmid DNA, DNA fragments, RNA, oligonucleotides, proteins, peptides, fluorogenic substances, cells, viruses, bacteria, chemical compounds, vaccines, etc.) or other biological samples as provided herein on a matrix comprised of a material that dissolves or dissociates in a solvent that allows complete recovery or substantial recovery (e.g., recovery of at least 50 percent, preferably at least 60 percent, more preferably at least 70 percent, more preferably at least 80 percent, and typically in more preferred embodiments at least 85 percent, more preferably at least 90, 91, 92, 93 or 94 percent, more preferably at least 95 percent, still more preferably greater than 96, 97, 98 or 99 percent) of the sample material. For example, a liquid matrix may be selected based on the properties of the matrix material and/or of the sample depending on the particular methodology being employed and in a manner that permits recovery of one or more desired structural or functional properties of the sample (e.g., biological activity). Similarly, as another example, the matrix material may dissociate in an appropriate solvent and may, but need not, become fully solubilized, such that a dispersion, suspension, colloid, gel, hydrogel, sap, slurry, syrup, or the like may be obtained.

In certain preferred embodiments at least one solvent for use in compositions and methods disclosed herein will be aqueous, for example, a biocompatible solvent such as a biological fluid, a physiological solution or an aqueous biological buffer solution selected to support a biological structure and/or function of a biomolecule by preserving for that biomolecule a favorable chemical milieu that is conducive to the structure and/or function. Non-limiting examples of such biocompatible solvents include physiological saline (e.g., approximately 145 mM NaCl), Ringer's solution, Hanks' balanced salt solution, Dulbecco's phosphate buffered saline, Erle's balanced salt solution, and other buffers and solutions and the like as will be known to those familiar with the art, including those containing additives as may be desired for particular biomolecules of interest.

According to other embodiments, however, the invention need not be so limited and other solvents may be selected, for instance, based on the solvent polarity/polarizability (SPP) scale value using the system of Catalan et al. (e.g., 1995 Liebigs Ann. 241; see also Catalan, 2001 In: Handbook of Solvents, Wypych (Ed.), Andrew Publ., NY, and references cited therein), according to which, for example, water has a SPP value of 0.962, toluene a SPP value of 0.655, and 2-propanol a SPP value of 0.848. Methods for determining the SPP value of a solvent based on ultraviolet measurements of the 2-N,N-dimethyl-7-nitrofluorene/2-fluoro-7-nitrofluorene probe/homomorph pair have been described (Catalan et al., 1995). Solvents with desired SPP values (whether as pure single-component solvents or as solvent mixtures of two, three, four or more solvents; for solvent miscibility see, e.g., Godfrey 1972, Chem. Technol. 2:359) based on the solubility properties of a particular matrix material can be readily identified by those having familiarity with the art in view of the instant disclosure.

Liquid Matrix

According to non-limiting theory, a liquid matrix as described herein, which may in preferred embodiments comprise a matrix material dissolved or dissociated in a biocompatible solvent, may comprise a polymer structure that, by forming a matrix (e.g., a spatially organized support or scaffold), creates a three dimensional space which allows constituent biological material of the biological sample to associate with the matrix. Further according to non-limiting theory, the dissolvable or dissociable matrix material may also be used in certain contemplated embodiments to spatially organize the introduction of stabilizing agents such as salts, sugars, inhibitors, buffers and/or other stabilizers. The matrix also allows inclusion of components (e.g., buffers) for the adjustment of pH and other parameters for optimal storage conditions, and may optionally comprise one or a plurality of detectable indicators as provided herein, such as color-based pH indicators, and/or other chemical indicators.

In certain preferred embodiments the matrix material comprises polyvinyl alcohol (PVA), a dissolvable matrix material. PVA may be obtained from a variety of commercial sources (e.g., Sigma-Aldrich, St. Louis, Mo.; Fluka, Milwaukee, Wis.) and is available in specific discrete molecular weights or, alternatively, as a polydisperse preparation of polymers within several prescribed molecular weight ranges based on variable degrees of polymerization. For example, the Mowiol® series of PVA products may be obtained from Fluka in approximate molecular weight ranges of 16, 27, 31, 47, 55, 61, 67, 130, 145, or 195 kDa, and other PVA products are known, such as the preparation having average molecular weight of 30-70 kDa (Sigma No. P 8136) as used in the accompanying Examples. Based on the present disclosure, the skilled person will appreciate that, depending on the physicochemical properties (e.g., molecular mass, hydrophobicity, surface charge distribution, solubility, etc.) of a particular biomolecule of interest that is present in a biological sample to be stored under liquid conditions as described herein, these or other PVA products, or other suitable matrix materials that dissolve or dissociate in a solvent, can be identified readily and without undue experimentation, for use according to the present compositions and methods.

As described herein, a matrix for substantially liquid storage of a biological sample may, according to certain embodiments, be prepared from a solution that comprises from about 0.1% to about 10% weight-to-volume PVA, which in certain related embodiments may comprise from about 0.5% to about 5%, about 1% to about 5%, about 0.5% to about 1.5%, about 1%, about 3%, or about 5% weight-to-volume PVA, where “about” may be understood to represent quantitative variation that may be more or less than the recited amount by less than 50%, more preferably less than 40%, more preferably less than 30%, and more preferably less than 20%, 15%, 10% or 5%. Similar weight-to-volume ratios and tolerances may pertain for other liquid matrix materials in at least some distinct embodiments wherein the matrix material is other than PVA.

According to certain other embodiments, the liquid matrix material may be any suitable material having the compatible characteristics for storing a particular type of biological sample in a manner that satisfactorily preserves the desired structural and/or functional properties, said characteristics including the ability to form a matrix within the interstices of which the biological molecules of interest are deposited, and whereby the matrix molecules do not interfere with one or more biological activities of interest in the sample.

Additional non-limiting examples of a liquid matrix material include polyvinyl pyrrolidone, carboxymethyl cellulose, 2-hydroxyethyl cellulose (C₂H₆O₂)ₓ, poly(2-ethyl-2-oxazoline) [—N(COC₂H₅)CH₂CH₂—]ₙ, polyvinyl alcohol, trehalose, polyethylene glycol, agarose, poly-N-vinylacetamide, polyvinylpyrrolidone, poly(4-vinylpyridine), polyphenylene oxide, reversibly crosslinked acrylamide, polymethacrylate, carbon nanotubes (e.g., Dyke et al., 2003 JACS 125:1156; Mitchell et al., 2002 Macromolecules 35:8825; Dagani, 2003 C&EN 81:5), polylactide, lactide/glycolide copolymer, hydroxymethacrylate copolymer, calcium pectinate, hydroxypropyl methylcellulose acetate succinate (e.g., Langer, 1990 Science 249:1527; Langer, 1993 Accounts Chem. Res. 26:537-542), heparin sulfate proteoglycan, hyaluronic acid, glucuronic acid (e.g., Kirn-Safran et al., 2004 Birth Defects Res. C. Embryo Today 72:69-88), thrombospondin-1 N-terminal heparin-binding domain (e.g., Elzie et al., 2004 Int. J. Biochem. Cell Biol. 36:1090; Pavlov et al., 2004 Birth Defects Res. C. Embryo Today 72:12-24), fibronectin (e.g., Wierzbicka-Patynowski et al., 2003 J Cell Sci. 116(Pt 16):3269-76), a peptide/water-soluble polymeric modifier conjugate (e.g., Yamamoto et al., 2002 Curr Drug Targets 3(2):123-30), and collagen or collagen fragments including basement membrane collagen peptides (e.g., Ortega et al., 2002 J Cell Sci. 115(Pt 22):4201-14).

Certain embodiments of the present invention are contemplated that expressly exclude liquid matrix materials such as soluble cationic polymers (e.g., DEAE-dextran) or anionic polymers (e.g., dextran sulphate) or agarose when used, absent other components of the herein described embodiments, with a di- or trisaccharide stabilizer (e.g., trehalose, lactitol, lactose, maltose, maltitol, sucrose, sorbitol, cellobiose, inositol, or chitosan) as disclosed for dry protein storage, for example, in one or more of U.S. Pat. No. 5,240,843, U.S. Pat. No. 5,834,254, U.S. Pat. No. 5,556,771, U.S. Pat. No. 4,891,319, U.S. Pat. No. 5,876,992, WO 90/05182, and WO 91/14773, but certain other embodiments of the present invention contemplate the use of such combinations of a liquid matrix material and at least one such first di- or trisaccharide stabilizer, along with a second stabilizer that comprises a biological or biochemical inhibitor which may be a trehalase inhibitor as described herein and having antimicrobial activity (e.g., validamycin A, suidatrestin, validoxylamine A, MDL 26537, trehazolin, salbostatin, and/or casuarine-6-O-α-D-glucopyranoside), which combination the cited documents fail to suggest. Certain other embodiments of the present invention contemplate the use of such combinations of a dissolvable or dissociatable matrix material and at least one such di- or trisaccharide stabilizer for substantially liquid storage of biological samples other than proteins, for example, polynucleotides such as DNA, RNA, synthetic oligonucleotides, genomic DNA, natural and recombinant nucleic acid plasmids and constructs, and the like.

In certain embodiments disclosed herein, a matrix for liquid or substantially liquid storage of a biological sample comprises at least one matrix material that comprises a polymer, and a stabilizer, wherein the polymer does not covalently self-assemble and has the structure:

—[—X—]ₙ—

wherein X is —CH₃, —CH₂—, —CH₂CH(OH)—, substituted —CH₂CH(OH)—, —CH₂CH(COOH)—, substituted —CH₂CH(COOH)—, —CH═CH₂, —CH═CH—, C₁-C₂₄ alkyl or substituted alkyl, C₂₋₂₄ alkenyl or substituted alkenyl, polyoxyethylene, polyoxypropylene, or a random or block copolymer thereof; and wherein n is an integer having a value of about 1-100, 101-500, 501-1000, 1001-1500, or 1501-3000. Synthesis of such polymers may be accomplished using reagents that are commercially available (e.g., PVA as discussed above or other reagents from SigmaAldrich or Fluka, or Carbopol® polymers from Noveon, Inc., Cleveland, Ohio, etc.) and according to established procedures, such as those found in Fiesers' Reagents for Organic Synthesis (T.-L. Ho (Ed.), Fieser, L. F. and Fieser, M., 1999 John Wiley & Sons, NY).

“Alkyl” means a straight chain or branched, noncyclic or cyclic, unsaturated or saturated aliphatic hydrocarbon containing from 1 to 10 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, and the like; while unsaturated cyclic alkyls include cyclopentenyl and cyclohexenyl, and the like. Cyclic alkyls are also referred to herein as “homocycles” or “homocyclic rings.” Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“Alkoxy” means an alkyl moiety attached through an oxygen bridge (i.e., —O-alkyl) such as methoxy, ethoxy, and the like.

“Alkylthio” means an alkyl moiety attached through a sulfur bridge (i.e., —S-alkyl) such as methylthio, ethylthio, and the like.

“Alkylsulfonyl” means an alkyl moiety attached through a sulfonyl bridge (i.e., —SO₂-alkyl) such as methylsulfonyl, ethylsulfonyl, and the like.

“Alkylamino” and “dialkylamino” mean one or two alkyl moieties attached through a nitrogen bridge (i.e., —N-alkyl) such as methylamino, ethylamino, dimethylamino, diethylamino, and the like.

“Aryl” means an aromatic carbocyclic moiety such as phenyl or naphthyl.

“Arylalkyl” means an alkyl having at least one alkyl hydrogen atom replaced with an aryl moiety, such as benzyl, —(CH₂)₂ phenyl, —(CH₂)₃ phenyl, —CH(phenyl)₂, and the like.

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl.

“Heteroarylalkyl” means an alkyl having at least one alkyl hydrogen atom replaced with a heteroaryl moiety, such as —CH₂ pyridinyl, —CH₂ pyrimidinyl, and the like.

“Halogen” means fluoro, chloro, bromo and iodo.

“Haloalkyl” means an alkyl having at least one hydrogen atom replaced with halogen, such as trifluoromethyl and the like.

“Heterocycle” (also referred to as a “heterocyclic ring”) means a 4- to 7-membered monocyclic, or 7- to 10-membered bicyclic, heterocyclic ring which is either saturated, unsaturated, or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include heteroaryls as defined above. Thus, in addition to the heteroaryls listed above, heterocycles also include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydropyrimidinyl, tetrahydrothiophenyl, tetrahydrothiopyranyl, tetrahydropyrimidinyl, tetrahydrothiophenyl, tetrahydrothiopyranyl, and the like.

“Heterocyclealkyl” means an alkyl having at least one alkyl hydrogen atom replaced with a heterocycle, such as —CH₂ morpholinyl, and the like.

“Homocycle” (also referred to herein as “homocyclic ring”) means a saturated or unsaturated (but not aromatic) carbocyclic ring containing from 3-7 carbon atoms, such as cyclopropane, cyclobutane, cyclopentane, cyclohexane, cycloheptane, cyclohexene, and the like.

The term “substituted” as used herein means any of the above groups (e.g., alkyl, alkenyl, alkynyl, homocycle) wherein at least one hydrogen atom is replaced with a substituent. In the case of a keto substituent (“—C(═O)—”) two hydrogen atoms are replaced. When substituted one or more of the above groups are substituted, “substituents” within the context of this invention include halogen, hydroxy, cyano, nitro, amino, alkylamino, dialkylamino, alkyl, alkoxy, alkylthio, haloalkyl, aryl, arylalkyl, heteroaryl, heteroarylalkyl, heterocycle and heterocyclealkyl, as well as —NRₐR_{b}, —NRₐC(═O)R_{b}—, NRₐC(═O)NRₐNR_{b}, —NRₐC(═O)OR_{b}—NRₐSO₂R_{b}, —C(═O)Rₐ, —C(═O)ORₐ, —C(═O)NRₐR_{b}, —OC(═O) NRₐR_{b}, —ORₐ, —SRₐ, —SORₐ, —S(═O)₂Rₐ, —OS(═O)₂Rₐ and —S(═O)₂ORₐ. In addition, the above substituents may be further substituted with one or more of the above substituents, such that the substituent is substituted alkyl, substituted aryl, substituted arylalkyl, substituted heterocycle or substituted heterocyclealkyl. Rₐ and R_{b} in this context may be the same or different and independently hydrogen, alkyl, haloalkyl, substituted aryl, aryl, substituted aryl, arylalkyl, substituted arylalkyl, heterocycle, substituted heterocycle, heterocyclealkyl or substituted heterocyclealkyl.

The polymer preferably comprises a plurality of hydrogen-bonding moieties which may be the same or different, each hydrogen-bonding moiety having one or more groups capable of forming a hydrogen bond with the same or different moieties, as may be present on a biomolecule of interest within a biological sample. Each hydrogen-bonding moiety may have hydrogen-bonding donor and/or acceptor groups. Preferably each hydrogen-bonding moiety has both donor and acceptor groups. However, it is possible for hydrogen-bonding moieties to have only donor or acceptor groups. Thus, for example, a polymer having hydrogen-bonding moieties with solely donor groups may be used together with a polymer having hydrogen-bonding moieties with solely acceptor groups. Also, for instance, one polymer may comprise both hydrogen-bonding moieties which are wholly donor groups and hydrogen-bonding moieties which are wholly acceptor groups.

Preferred polymers additionally have some monomeric units having only one hydrogen bonding group. Such mono-functional monomers are present as chain stoppers and can be used to control the molecular weight of the polymer. It is preferable if these mono-functional monomers are present at 10% or less of the total number of monomeric material comprising the polymer, more preferably less than 5%. The polymers according to the present invention which contain one or more hydrogen bonding groups are also referred to as “capable of forming at least one hydrogen bond” and may be capable of doing so with other polymer molecules, with at least one stabilizer and/or with at least one biomolecule of interest that is present in a biological sample, for instance, a nucleic acid molecule or a polypeptide molecule. The strength of each hydrogen bond preferably varies from 1-40 kcal/mol, depending on the nature and functionality of the donor and acceptors involved. The groups in the hydrogen-bonding moieties which are preferably capable of forming a hydrogen bond with the same or different moieties are provided in the form of “substituted X” moieties and may suitably be selected from, for example, >C═O, —COO—, —COOH, —O—, —O—H, —NH₂, >N—H, >N—, —CONH—, —F, —C═N— groups and mixtures thereof. Preferably the groups are selected from >C═O, —O—H, —NH₂, >NH, —CONH—, —C═N— and mixtures thereof.

Preferably the polymer molecules may be capable of forming at least one hydrogen bond with a component of the biological sample in a manner that is preferential to polymer-polymer hydrogen bond formation, but these invention embodiments are not so limited so long as the polymer does not covalently self-assemble. According to non-limiting theory, stabilizing interactions among the biological sample, the matrix and/or the stabilizer result from hydrogen-bonding interactions. However, other non-covalent forces may also contribute to the bonding such as, for example, ionic bonds, electrostatic forces, van der Waal's forces, metal coordination, hydrophobic forces and, when the hydrogen-bonding moieties comprise one or more aromatic rings, pi-pi stacking (Russell, J B. 1999. General Chemistry. Second Edition. McGraw-Hill, Columbus, Ohio; Lodish et al. (Eds.) 2000. Molecular Cell Biology. Fourth Edition. W.H. Freeman).

As described herein, according to certain embodiments, the polymer is capable of non-covalent association with one or more stabilizers, and according to certain other non-limiting embodiments, the polymer is capable of non-covalent association with one or more molecular species present in the liquid-storable biological sample and having origins in the subject or biological source (e.g., biomolecules such as polypeptides, polynucleotides, naturally occurring oligosaccharides, naturally occurring lipids, and the like). Methodologies and instrumentation for the determination of non-covalent associations between such components will be known to those familiar with the art in view of the present disclosure, and may include techniques such as electrospray ionization mass spectrometry (Loo et al., 1989 Anal. Biochem. June; 179(2):404-412; Di Tullio et al. 2005J. Mass Spectrom. Jul; 40(7):845-865), diffusion NMR spectroscopy (Cohen et al., 2005 Angew Chem Int Ed Engl. Jan 14; 44(4):520-554), or other approaches by which non-covalent associations between molecular species of interest can be demonstrated readily and without undue experimentation (for example, circular dichroism spectroscopy, scanning probe microscopy, spectrophotometry and spectrofluorometry, and nuclear magnetic resonance of biological macromolecules; see e.g., Schalley C A et al. (Eds.) Analytical Methods in Supramolecular Chemistry, 2007, Wiley Publishers, Hoboken, N.J.; Sauvage and Hosseini (Eds.), Comprehensive Supramolecular Chemistry, 1996 Elsevier Science, Inc., New York, London, Tokyo; Cragg, P J (Ed.), A Practical Guide to Supramolecular Chemistry, 2005 Wiley & Sons, Ltd., West Sussex, UK; James et al. (Eds.), 2001 and 2005, Nuclear Magnetic Resonance of Macromolecules: Methods in Enzymology (vols. 338, 399 and 394) Academic Press, Ltd., London, UK).

### Stabilizer

The liquid sample matrix, according to certain preferred embodiments, may also be prepared (e.g., in the sample storage device) in a manner such that one or more wells contain at least one stabilizer, and in certain embodiments at least two stabilizers, which may include any agent that may desirably be included to preserve, stabilize, maintain, protect or otherwise contribute to the recovery (e.g., from the biological sample storage device) of a biological sample that has substantially the same biological activity as was present prior to the step of contacting the sample with the liquid matrix. The stabilizer may in certain embodiments comprise an agent that is a biological inhibitor or a biochemical inhibitor, as provided herein. Accordingly, in certain preferred embodiments the liquid matrix comprises at least one stabilizer that is such an inhibitor, for example, an anti-microbial agent such as (but not limited to) an anti-fungal and/or antibacterial agent capable of inhibiting or suppressing bacterial or fungal growth, viability and/or colonization, to inhibit microbial contamination of the stored sample during long-term storage. Stabilizers which may also be useful in the methods of this invention include polycations (see for example Slita et al., J Biotechnol. 2007 Jan. 20; 127(4):679-93. Epub 2006 Jul. 27), reducing agents (for example, dithiothreitol; Scopes, R. K. 1994 Protein Purification: Principals and Practices. Third edition, Springer, Inc., New York), steric stabilizers (such as alkyl groups, PEG chains, polysaccharides, alkyl amines; U.S. Pat. No. 7,098,033), small molecules, and amino acids (see for example U.S. Pat. No. 7,011,825), and buffers (Scopes, R. K. 1994 Protein Purification: Principals and Practices. Third edition, Springer, Inc., New York; Current Protocols, Protein Sciences, Cell Biology, Wiley and Sons, 2003). The stabilizer may in certain embodiments comprise a salt, glycerol, a detergent, a polyol, an osmolyte, a chaotrope, an organic solvent, an eletrostatic reagent, a metal ion, a ligand, an inhibitor, a cofactor or substrate, a chaperonin, a redox buffer, disulfide isomerase or a protease inhibitor, which may facilitate dissolution of certain biological samples, such as proteins (see for example U.S. Pat. No. 6,057,159; Scopes, R. K. 1994 Protein Purification Principals and Practices. Third edition, Springer, Inc., New York; Current Protocols, Protein Sciences, Cell Biology, Wiley and Sons, 2003).

Preferred stabilizers according to certain embodiments described herein comprise biological or biochemical inhibitors that are glycosidase inhibitors, such as trehalase inhibitors (e.g., suidatrestin, validamycin A, validoxylamine A, MDL 26537, trehazolin, salbostatin, casuarine-6-O-α-D-glucopyranoside) described by Asano (2003 Glycobiol. 13(10):93R-104R), Knuesel et al. (1998 Comp. Biochem. Physiol. B Biochem. Mol. Biol. 120:639), Dong et al. (2001 J. Am. Chem. Soc. 123(12):2733) and Kameda et al. (1980 J. Antibiot. (Tokyo) 33(12):1573). An unexpected advantage associated with the use of such inhibitors in these invention embodiments derives from antimicrobial properties of these inhibitors, in addition to their biomolecule-stabilizing effects which are believed, according to non-limiting theory, to derive from non-covalent interactions, such as hydrogen bonding, between the inhibitor and one or more of the biomolecule in the biological sample, the matrix material and/or the solvent.

In other embodiments, a stabilizer may be another glycosidase inhibitor such as a chitinase inhibitor (e.g., allosamidin, argifin, argadin), an α-glucosidase inhibitor (e.g., valiolamine, voglibose, nojirimycin, 1-deoxynojirimycin, miglitol, salacinol, kotalanol, NB-DNJ, N,N-DNJ, glycovir, castanospermine), a glycogen phosphorylase inhibitor (e.g., D-ABI, isofagomine, fagomine), a neuraminidase inhibitor (e.g., DANA, FANA, 4-amino-4-deoxy-DANA, zanamivir, BCX 140, GS 4071, GS 4104, peramivir), a ceramide glucosyltransferase inhibitor or a lysosomal glycosidase inhibitor, non-limiting examples of all of which glycosidase inhibitors are described by Asano (2003 Glycobiol. 13(10):93R-104R).

In certain related embodiments the stabilizer which comprises a biological inhibitor or a biochemical inhibitor may be a reducing agent, an alkylating agent, an antimicrobial agent, a kinase inhibitor, a phosphatase inhibitor, a caspase inhibitor, a granzyme inhibitor, a cell adhesion inhibitor, a cell division inhibitor, a cell cycle inhibitor, a small molecule inhibitor, a lipid signaling inhibitor and/or a protease inhibitor. Those familiar with the art will be aware of a wide range of readily available inhibitors that may be selected depending on the nature of the biological sample and the particular bioactivity of interest. See, e.g., Calbiochem® Inhibitor SourceBook™ (2004 (1st Ed.) and 2007 (2^{nd} Ed.), EMD Biosciences, La Jolla, Calif.). For antimicrobial agents, see, e.g., Pickering, L K, Ed. 2003 Red Book: Report of the Committee on Infectious Diseases, 26^{th} edition. Elk Grove Village, Ill., pp. 695-97.; American Academy of Pediatrics, 1998, Pediatrics, 101(1), supplement; Disinfection Sterilization and Preservation, Seymour S. Block (Ed.), 2001 Lippincott Williams & Wilkins, Philadelphia; Antimicrobial Inhibitors, A. I. Laskin and H. A. Lechevalier, (Eds.), 1988 CRC Press, Boca Raton, Fla.; Principles and Practice of Disinfection, Preservation and Sterilization, A. D. Russell et al., (Eds.), 1999, Blackwell Science, Malden, Mass.; Antimicrobial/anti-infective materials, S. P. Sawan et al., (Eds.), 2000 Technomic Pub. Co., Lancaster, Pa.; Development of novel antimicrobial agents: emerging strategies, K. Lohner, (Ed.), 2001 Wymondham, Norfolk, UK; Conte, J. E. Manual of antibiotics and infectious diseases (9^{th} Ed.), 2001, Lippincott Williams & Wilkins, Philadelphia.

As noted above, in certain preferred embodiments the stabilizer may be a trehalase inhibitor such as the fungizide validamycin A (e.g., Kameda et al., 1980 J. Antibiot. (Tokyo) 33(12):1573; Dong et al., 2001 J. Am. Chem. Soc. 123(12):2733; available from Research Products International Corp., Mt. Prospect, Ill., catalog no. V21020), and in certain other embodiments the stabilizer, for instance, a stabilizer that comprises an inhibitor that is a biological inhibitor or a biochemical inhibitor, may be a protease inhibitor such as TL-3 (Lee et al., 1998 Proc. Nat. Acad. Sci. USA 95:939; Lee et al., 1999 J. Amer. Chem. Soc. 121:1145; Buhler et al., 2001 J. Virol. 75:9502), N-α-tosyl-Phe-chloromethylketone, N-α-tosyl-Lys-chloromethylketone, aprotinin, phenylmethylsulfonyl fluoride or diisopropylfluoro-phosphate, or a phosphatase inhibitor such as sodium orthovanadate or sodium fluoride.

As described herein, an added advantage of the liquid matrix is that the storage container can be directly used as a reaction chamber. The stability and activity of proteins in liquid form may be dependent on activity requirements such as pH, salt concentration, and cofactors.

Biological material provided in or derived from a biological sample may be added to the wells or tubes in combination with the liquid matrix in liquid form (e.g., by simultaneously contacting the sample well with the sample and the matrix dissolved or dissociated in a solvent). The liquid matrix does not, in preferred embodiments, interfere with biochemical reactions such that purification steps may not be required to separate the matrix from the biological sample prior to further processing of the sample, for instance, prior to performance of biochemical reactions, such as assays or the like, in the wells of the sample storage device. However, if purification is required, the liquid matrix can be removed from the sample using techniques well known to those in the art, such as filtration, centrifugation, ion exchange, size exclusion, chromatography, or phase separation, or other purification methods known to those persons trained in the relevant art.

Accordingly, certain embodiments of the invention expressly contemplate a biological sample storage device that does not include trehalose as a component of a sample well or of a matrix material, and similarly certain embodiments may expressly exclude from the sample well or matrix material the presence of polystyrene and/or of hydroxyectoine. In view, however, of the unexpected advantages disclosed herein as they relate to the inclusion of a trehalase inhibitor such as validamycin (e.g., validamycin A, or other trehalase inhibitors described herein) as an inhibitor in biological sample storage devices, certain other embodiments contemplated herein may include a first stabilizer that may be any one or more of trehalose, lactitol, lactose, maltose, maltitol, mannitol, sucrose, sorbitol, cellobiose, inositol, chitosan, hydroxyectoine, and/or polystyrene, provided a second stabilizer that is a trehalase inhibitor as provided herein is also present, for example a trehalase inhibitor selected from suidatrestin, validamycin A, validoxylamine A, MDL 26537, trehazolin, salbostatin, and casuarine-6-O-α-D-glucopyranoside. According to non-limiting theory, a trehalase inhibitor known to the agricultural art as a fungicide (e.g., validamycin A), provides a surprising stabilizing effect when used in combination with a liquid matrix in the biological sample storage devices, as disclosed herein. Alternatively or additionally to the use disclosed herein of validamycin (or another trehalase inhibitor) along with the dissolvable matrix, other small molecules that have activity as inhibitors or activators of trehalase may be usefully included in the storage devices, as additional stabilizers or as additives to the matrix material and/or to the sample, including natural disaccharides, pseudo-sugars that are also known as carba-sugars, and/or other inhibitors/activators of trehalase. In addition, trehalase inhibitors such as validamycin provide an advantage according to certain embodiments disclosed herein, in that they protect the long-term storage media from fungal, bacterial or other types of undesirable microbial contamination.

Additional stabilizers contemplated for use according to certain other embodiments of the present invention may be present in a liquid sample matrix but are not covalently linked to the polymeric matrix material as disclosed herein, and may include small molecules that comprise structures (i)-(xv), including several known amino acid side chains and mono-, di- and polysaccharides such as:

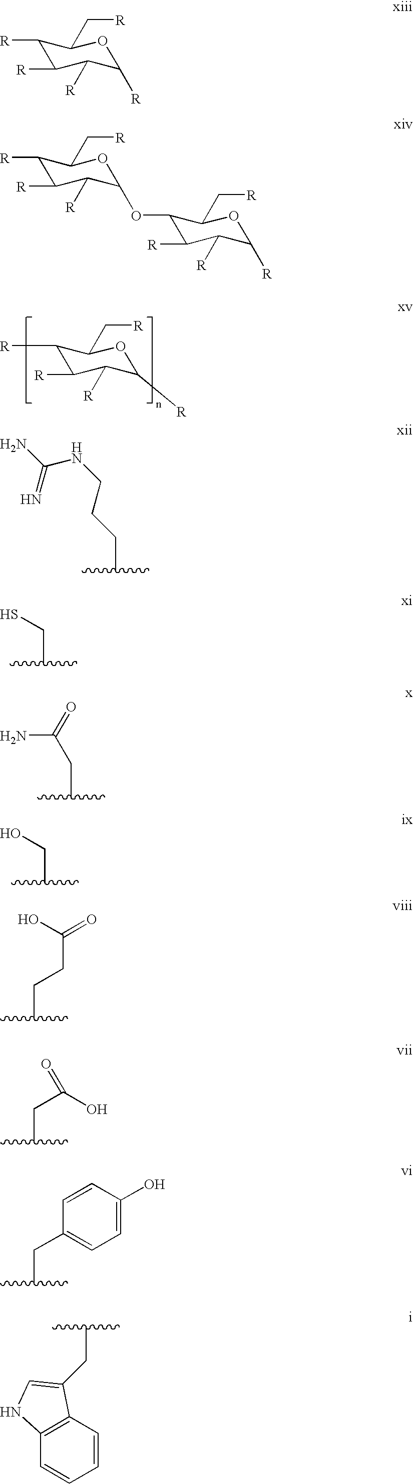

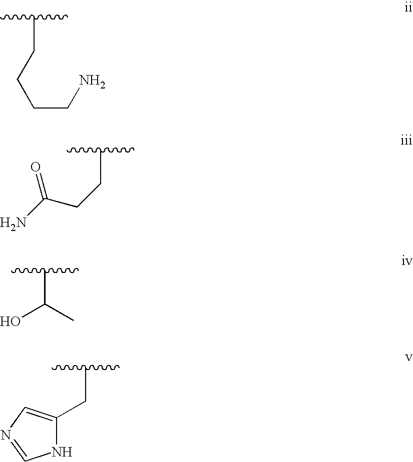

wherein R is selected from —H, —OH, —CH₂OH, —NHAc and —OAc. Such compositions are known in the art and are readily available from commercial suppliers. Additional stabilizers contemplated for use according to certain other embodiments of the present invention may also include D-(+)-raffinose, β-gentiobiose, ectoine, D-(+)-raffinose pentahydrate, myo-inositol, hydroxyectoine, magnesium D-gluconate, 2-keto-D-gluconic acid hemicalcium salt hydrate, D(+)-melezitose, calcium lactobionate monohydrate, β-lactose, turanose, and D-maltose.

### Detectable Indicator

Detectable indicators include compositions that permit detection (e.g., with statistical significance relative to an appropriate control, as will be know to the skilled artisan) or similar determination of any detectable parameter that directly relates to a condition, process, pathway, induction, activation, inhibition, regulation, dynamic structure, state, contamination, degradation or other activity or functional or structural change in a biological sample, including but not limited to altered enzymatic (including proteolytic and/or nucleolytic), respiratory, metabolic, catabolic, binding, catalytic, allosteric, conformational, or other biochemical or biophysical activity in the biological sample, and also including interactions between intermediates that may be formed as the result of such activities, including metabolites, catabolites, substrates, precursors, cofactors and the like.

A wide variety of detectable indicators are known to the art and can be selected for inclusion in the presently disclosed compositions and methods depending on the particular parameter or parameters that may be of interest for particular biological samples in particular sample storage applications. Non-limiting examples of parameters that may be detected by such detectable indicators include detection of the presence of one or more of an amine, an alcohol, an aldehyde, a thiol, a sulfide, a nitrite, avidin, biotin, an immunoglobulin, an oligosaccharide, a nucleic acid, a polypeptide, an enzyme, a cytoskeletal protein, a reactive oxygen species, a metal ion, pH, Na⁺, K⁺, Cl⁻, a cyanide, a phosphate, selenium, a protease, a nuclease, a kinase, a phosphatase, a glycosidase, and a microbial contaminant, and others.

Examples of a broad range of detectable indicators (including colorimetric indicators) that may be selected for specific purposes are described in Haugland, 2002 Handbook of Fluorescent Probes and Research Products—Ninth Ed., Molecular Probes, Eugene, Oreg.; in Mohr, 1999 J. Mater. Chem., 9: 2259-2264; in Suslick et al., 2004 Tetrahedron 60:11133-11138; and in U.S. Pat. No. 6,323,039. (See also, e.g., Fluka Laboratory Products Catalog, 2001 Fluka, Milwaukee, Wis.; and Sigma Life Sciences Research Catalog, 2000, Sigma, St. Louis, Mo.) A detectable indicator may be a fluorescent indicator, a luminescent indicator, a phosphorescent indicator, a radiometric indicator, a dye, an enzyme, a substrate of an enzyme, an energy transfer molecule, or an affinity label. In certain preferred embodiments the detectable indicator may be one or more of phenol red, ethidium bromide, a DNA polymerase, a restriction endonuclease (e.g., a restriction enzyme used as a restriction nuclease such as a site- or sequence-specific restriction endonuclease), cobalt chloride (a moisture indicator that changes from blue color when water is present to pink when dry), Reichardt's dye (Aldrich Chemical) and a fluorogenic protease substrate.

A detectable indicator in certain embodiments may comprise a polynucleotide polymerase and/or a suitable oligonucleotide, either or both of which may be employed as an indicator or, in certain other embodiments, as components of other nucleic acids-based applications of the compositions and methods described herein. Polymerases (including DNA polymerases and RNA polymerases) useful in accordance with certain embodiments of the present invention include, but are not limited to, Thermus thermophilus (Tth) DNA polymerase, Thermus aquaticus (Taq) DNA polymerase, Thermologa neopolitana (Tne) DNA polymerase, Thermotoga maritima (Tma) DNA polymerase, Thermococcus litoralis (Tli or VENT™) DNA polymerase, Pyrococcus furiosus (Pfu) DNA polymerase, DEEPVENT™ DNA polymerase, Pyrococcus woosii (Pwo) DNA polymerase, Bacillus sterothermophilus (Bst) DNA polymerase, Bacillus caldophilus (Bca) DNA polymerase, Sulfolobus acidocaldarius (Sac) DNA polymerase, Thermoplasma acidophilum (Tac) DNA polymerase, Thermus flavus (Tfl/Tub) DNA polymerase, Thermus ruber (Tru) DNA polymerase, Thermus brockianus (DYNAZYME™) DNA polymerase, Methanobacterium thermoautotrophicum (Mth) DNA polymerase, mycobacterium DNA polymerase (Mtb, Mlep), and mutants, and variants and derivatives thereof. RNA polymerases such as T3, T5 and SP6 and mutants, variants and derivatives thereof may also be used in accordance with the invention.

Polymerases used in accordance with the invention may be any enzyme that can synthesize a nucleic acid molecule from a nucleic acid template, typically in the 5′ to 3′ direction. The nucleic acid polymerases used in the present invention may be mesophilic or thermophilic, and are preferably thermophilic. Preferred mesophilic DNA polymerases include T7 DNA polymerase, T5 DNA polymerase, Klenow fragment DNA polymerase, DNA polymerase III and the like. Preferred thermostable DNA polymerases that may be used in the methods of the invention include Taq, Tne, Tma, Pfu, Tfl, Tth, Stoffel fragment, VENT™ and DEEPVENT™ DNA polymerases, and mutants, variants and derivatives thereof (U.S. Pat. No. 5,436,149; U.S. Pat. No. 4,889,818; U.S. Pat. No. 4,965,188; U.S. Pat. No. 5,079,352; U.S. Pat. No. 5,614,365; U.S. Pat. No. 5,374,553; U.S. Pat. No. 5,270,179; U.S. Pat. No. 5,047,342; U.S. Pat. No. 5,512,462; WO 92/06188; WO 92/06200; WO 96/10640; Barnes, W. M., Gene 112:29-35 (1992); Lawyer et al., PCR Meth. Appl. 2:275-287 (1993); Flaman et al., Nucl. Acids Res. 22(15):3259-3260 (1994)).

Other detectable indicators for use in certain embodiments contemplated herein include affinity reagents such as antibodies, lectins, immunoglobulin Fc receptor proteins (e.g., Staphylococcus aureus protein A, protein G or other Fc receptors), avidin, biotin, other ligands, receptors or counterreceptors or their analogues or mimetics, and the like. For such affinity methodologies, reagents for immunometric measurements, such as suitably labeled antibodies or lectins, may be prepared including, for example, those labeled with radionuclides, with fluorophores, with affinity tags, with biotin or biotin mimetic sequences or those prepared as antibody-enzyme conjugates (see, e.g., Weir, D. M., Handbook of Experimental Immunology, 1986, Blackwell Scientific, Boston; Scouten, W. H., 1987 Methods in Enzymology 135:30-65; Harlow and Lane, Antibodies: A Laboratory Manual, 1988 Cold Spring Harbor Laboratory, Cold Spring Harbor, N.Y.; Haugland, Handbook of Fluorescent Probes and Research Products—Ninth Ed., 2002 Molecular Probes, Eugene, Oreg.; Scopes, R. K., Protein Purification: Principles and Practice, 1987, Springer-Verlag, NY; Hermanson, G. T. et al., Immobilized Affinity Ligand Techniques, 1992, Academic Press, Inc., NY; Luo et al., 1998 J. Biotechnol. 65:225 and references cited therein).

Certain other embodiments of the present invention relate to compositions and methods for liquid storage of a biological sample wherein the matrix contains at least one, and in certain related embodiments two, three, four, five, six, seven, eight, nine, ten or more detectable indicators, each of which comprises a unique and readily identifiable gas chromatography/mass spectrometry (GCMS) tag molecule. Numerous such GCMS tag molecules are known to the art and may be selected for use alone or in combination as detectable identifier moieties, for instance, to encode unique GCMS spectrometric profiles for separate storage matrices in distinct sample storage device wells. By way of illustration and not limitation, various different combinations of one, two or more such GCMS tags may be added to individual wells in a manner that permits each well to be identified on the basis of the GCMS “signature” of its contents, thereby permitting any sample that is subsequently removed from a storage device well to be traced back to its well of origin for identification purposes. Examples of GCMS tags include α,α,α-trifluorotoluene, α-methylstyrene, o-anisidine, any of a number of distinct cocaine analogues or other GCMS tag compounds having readily identifiable GCMS signatures under defined conditions, for instance, as are available from SPEX CertiPrep Inc. (Metuchen, N.J.) or from SigmaAldrich (St. Louis, Mo.), including Supelco® products described in the Supelco® 2005 gas chromatography catalog and available from SigmaAldrich.

The dissolvable (or dissociable) matrix may be applied to storage containers for biological samples, for example, by contacting or administering a matrix material that dissolves or dissociates in a solvent to one or a plurality of sample wells of a storage device as described herein. Biological material provided in or derived from a biological sample may also be added to the wells or tubes in combination with the storage matrix in liquid form (e.g., by simultaneously contacting the sample well with the sample and the matrix dissolved or dissociated in a solvent). The dissolvable matrix does not, in preferred embodiments, interfere with biochemical reactions such that purification steps may not be required to separate the matrix from the biological sample prior to further processing of the sample, for instance, prior to performance of biochemical reactions, such as assays or the like, in the wells of the sample storage device.

The buffer conditions in the dissolvable matrix may be adjusted such that greater than at least 90 percent, preferably greater than 95 percent, more preferably greater than 96, 97, 98 or 99 percent of the biological activity (e.g., enzymatic or affinity activity, or structural integrity or other biological activity as described herein and known to the art) of the biological sample is maintained, eliminating the need to laboriously remove the sample from the storage container and transfer it to a reaction buffer in a separate container. Certain such invention embodiments correspondingly provide the unexpected advantage of eliminating the need to separately aliquot and/or calibrate certain biological reagents each time a stored sample is to be assayed.

The matrix material may be treated for the storage and preservation of biological materials. It is well documented that the adjustment of buffer conditions and the addition of chemicals and enzymes and other reagents can stabilize DNA and RNA (for example, Sambrook, J., Fritsch, E. F. and Maniatis, T. (1989) Molecular Cloning: A Laboratory Manual, Cold Spring Harbor Laboratory Press, New York; Current Protocols, Nucleic Acid Chemistry, Molecular Biology, Wiley and Sons, 2003) and/or proteins, enzymes and/or other biological materials (for example, blood, tissue, bodily fluids) against degradation from enzymes, proteases and environmental factors (for example, Current Protocols, Protein Sciences, Cell Biology, Wiley and Sons, 2003). Matrix compositions for liquid storage and methods for their use that combine certain chemical components to provide beneficial effects on the biological sample are also contemplated and may vary according to particular samples and uses thereof.

Various such chemical components may include but are not limited to a buffer capable of maintaining a desired pH level as may be selected by those familiar with the art, for example, buffers comprising Tris, citrate, acetate, phosphate, borate, HEPES, MES, MOPS, PIPES, carbonate and/or bicarbonate or other buffers (see, e.g., Calbiochem® Biochemicals & Immunochemicals Catalog 2004/2005, pp. 68-69 and pages cited therein, EMD Biosciences, La Jolla, Calif.) and suitable solutes such as salts (e.g., KCl, NaCl, CaCl₂, MgCl₂, etc.) for maintaining, preserving, enhancing, protecting or otherwise promoting one or more biological sample components (e.g., biomolecules), or activity buffers that may be selected and optimized for particular activities of specific biomolecules such as nucleic acid hybridization or activities of enzymes, antibodies or other proteins, or other buffers, for instance, Tris buffer (THAM, Trometanol, 2-amino-2-(hydroxymethyl)-1,3-propane diol), Tris-EDTA buffer (TE), sodium chloride/sodium citrate buffer (SSC), MOPS/sodium acetate/EDTA buffer (MOPS), ethylenediamine tetraacetic acid (EDTA), sodium acetate buffer at physiological pH, and the like.

Additional chemical components that may beneficially enhance the recovery of biological activity from a liquid-storable biological sample also include but are not limited to solutes that may be included in the biocompatible solvent of which the liquid matrix is comprised (along with the matrix material), where such solutes provide a desired isotonic, hypertonic or hypotonic environment as may be selected by those familiar with the art, for example an isotonic saline solution to prevent disruption of cellular membranes. Hence, as will be appreciated by those skilled in the art in view of the present disclosure, depending on the particular biological sample to be stored in liquid form and on the particular biological activity to be recovered from such sample, it may be desirable to formulate the liquid matrix with a biocompatible solvent that is isotonic, hypertonic or hypotonic relative to the sample.

By way of background, osmotic shock results from exposure of cells to solutions of different osmotic pressures, where osmosis involves the net diffusion of water across a selectively permeable membrane that is permeable in both directions to water, but varyingly permeable to solutes, wherein the water diffuses from one solution into another of lower water potential. The osmotic pressure of a solution is the pressure which must be exerted upon it to prevent passage of distilled water into it across a semipermeable membrane (i.e., a membrane that is impermeable to all solutes, but is freely permeable to solvent), and is often measured in Pascals (1 Pa=1 Newton/m²). Conversely, water potential is the net tendency of any system to give up water to its surroundings. As the water potential of pure water at atmospheric pressure is, by definition, zero pressure units, any addition of solute to pure water reduces its water potential and makes its value negative. Thus, water movement is from a system with higher (i.e., less negative) water potential to one with lower (i.e., more negative) water potential.

Hence, according to certain herein disclosed embodiments, a liquid-storable biological sample may comprise a suspension of cells in a hypertonic liquid matrix, such as one formulated with a biocompatible solvent having a solute concentration that is higher than that inside the cells present in the liquid solution, thus causing water to diffuse out of the cells. In these and related embodiments a hypertonic liquid matrix is provided having a greater relative solute concentration when compared to the solute concentration of a membrane-boundaried liquid compartment such as that within the cell (e.g., the cytosol is hypotonic relative to the liquid matrix). Such a hypertonic solution has a lower water potential than a solution that is hypotonic to it and has a correspondingly greater osmotic pressure. Thus, for instance, a hypotonic solution has a solute concentration that is lower than the solute concentration inside cells suspended in that solution, and therefore causes water to diffuse into the cells. A hypotonic solution has a lower relative solute concentration (i.e., higher water potential) than another solution. Certain other embodiments relate to liquid matrix formulations that may be isotonic solutions that have solute concentrations that are equal to intracellular solute concentrations (i.e., as indicated by their osmotic pressure). Separation of isotonic solutions by selectively permeable membranes (e.g., cell membranes) results in no net passage of water in either direction across the cell membrane, since the solutions have the same water potential.

Other chemical components that may be included in liquid storage matrices include ethylenediamine tetraacetic acid (EDTA), human placental ribonuclease inhibitor, bovine ribonuclease inhibitor, porcine ribonuclease inhibitor, diethyl pyrocarbonate, ethanol, formamide, guanidinium thiocyanate, vanadyl-ribonucleoside complexes, macaloid, proteinase K, heparin, hydroxylamine-oxygen-cupric ion, bentonite, ammonium sulfate, dithiothreitol (DTT), beta-mercaptoethanol or specific inhibiting antibodies.

Accordingly, certain invention embodiments contemplate a matrix for liquid storage of a biological sample, comprising a matrix material that dissolves or dissociates in a solvent, at least one stabilizer, and a sample treatment composition. The sample treatment composition may comprise an activity buffer as described below, and/or the sample treatment composition may comprise one or more of a cell lysis buffer, a free radical trapping agent, a sample denaturant, and a pathogen-neutralizing agent. As provided by these embodiments, the liquid storage matrix may thus comprise a set of components prepared to effect a desired treatment on a biological sample when the sample is introduced to the matrix, for example, in embodiments wherein the step of contacting the sample with the matrix occurs simultaneously with, or immediately following dissolving or dissociating the matrix material in the buffer.

An activity buffer may comprise a solvent or solution in liquid form, including a concentrate, which is suitable for a desired use of the biological sample stored in liquid matrix, such as a functional or structural characterization of one or more components of the sample.

Non-limiting examples of such uses may include determining one or more enzyme activities, determining intermolecular binding interactions, detecting the presence of a specific polynucleotide or amino acid sequence or of an immunologically defined epitope or of a defined oligosaccharide structure, detection of particular viruses or of microbial cells or of human or animal cells, determining particular metabolites or catabolites, etc., all of which can be accomplished using conditions that are defined and known to those skilled in the relevant art, including suitable conditions that can be provided through contacting the sample with an appropriate activity buffer.

A cell lysis buffer may be any composition that is selected to lyse (i.e., disrupt a boundary membrane of) a cell or organelle, and many such formulations are known to the art, based on principles of osmotic shock (e.g., hypotonic shock) and/or disruption of a cell membrane such as a plasma membrane through the use of a surfactant such as a detergent (e.g., Triton® X-100, Nonidet® P-40, sodium dodecyl sulfate, deoxycholate, octyl-glucopyranoside, betaines, or the like) and/or solute (e.g., urea, guanidine hydrochloride, guanidinium isothiocyanate, high salt concentration) system. Numerous cell lysis buffers are known and can be appropriately selected as a function of the nature of the biological sample and of the biomolecule(s), biological activities or biological structures that are desirably recovered, which may also in some embodiments include the selection of appropriate pH buffers, biological or biochemical inhibitors and detectable indicators.

Sample denaturants similarly may vary as a function of the biological sample and the liquid storage matrix, but may include an agent that non-covalently alters (e.g., with statistical significance relative to an appropriate control such as an untreated sample) at least one of the three-dimensional conformation, quarternary, tertiary and/or secondary structure, degree of solvation, surface charge profile, surface hydrophobicity profile, or hydrogen bond-forming capability of a biomolecule of interest in the sample. Examples of sample denaturants include chaotropes (e.g., urea, guanidine, thiocyanate salts), detergents (e.g., sodium dodecyl sulfate), high-salt conditions or other agents or combinations of agents that promote denaturing conditions.

Free radical trapping agents for use in certain embodiments may include any agent that is capable of stably absorbing an unpaired free radical electron from a reactive compound, such as reactive oxygen species (ROS), for example, superoxide, peroxynitrite or hydroxyl radicals, and potentially other reactive species, and antioxidants represent exemplary free radical trapping agents. Accordingly a wide variety of known free radical trapping agents are commercially available and may be selected for inclusion in certain embodiments of the presently disclosed compositions and methods. Examples include ascorbate, beta-carotene, vitamin E, lycopene, tert-nitrosobutane, alpha-phenyl-tert-butylnitrone, 5,5-dimethylpyrroline-N-oxide, and others, as described in, e.g., Halliwell and Gutteridge (Free Radicals in Biology and Medicine, 1989 Clarendon Press, Oxford, UK, Chapters 5 and 6); Vanin (1999 Meth. Enzymol. 301:269); Marshall (2001 Stroke 32:190); Yang et al. (2000 Exp. Neurol. 163:39); Zhao et al. (2001 Brain Res. 909:46); and elsewhere.

As noted above, certain embodiments contemplate inclusion of a pathogen-neutralizing agent in the presently disclosed compositions and methods, which includes any agent that is capable of completely or partially, but in any event in a manner having statistical significance relative to an appropriate control, neutralizing, impairing, impeding, inhibiting, blocking, preventing, counteracting, reducing, decreasing or otherwise blocking any pathogenic effect of a pathogen such as a bacterium, virus, fungus, parasite, prion, yeast, protozoan, infectious agent or any other microbiological agent that causes a disease or disorder in humans or vertebrate animals. Persons familiar with the relevant art will recognize suitable pathogen-neutralizing agents for use according to the present disclosure. Exemplary agents include sodium azide, borate, sodium hypochlorite, hydrogen peroxide or other oxidizing agents, sodium dichloroisocyanurate, ethanol, isopropanol, antibiotics, fungicides, nucleoside analogues, antiviral compounds, and other microbicides; these or others may be selected according to the properties of the particular biological sample of interest.

Provided herein are embodiments directed to kits that comprise the biological sample storage device as described herein, along with one or more ancillary reagents that may be selected for desired uses. Optionally the kit may also include a box, case, jar, drum, drawer, cabinet, carton, carrier, handle, rack, tray, pan, tank, bag, envelope, sleeve, housing or the like, such as any other suitable container. Ancillary reagents may include one or more solvents or buffers as described herein and known to the art, and may in certain embodiments include an activity buffer.

It is contemplated that the present invention will be of major value in high throughput screening; i.e., in automated testing or screening of a large number of biological samples. It has particular value, for example, in screening synthetic or natural product libraries for active compounds. The apparatus and methods of the present invention are therefore amenable to automated, cost-effective high throughput biological sample testing or drug screening and have immediate application in a broad range of pharmaceutical drug development programs. Typically, and in certain preferred embodiments such as for high throughput drug screening, candidate agents are provided as “libraries” or collections of compounds, compositions or molecules. Such molecules typically include compounds known in the art as “small molecules” and having molecular weights less than 10⁵ daltons, preferably less than 10⁴ daltons and still more preferably less than 10³ daltons. Candidate agents further may be provided as members of a combinatorial library, which preferably includes synthetic agents prepared according to a plurality of predetermined chemical reactions performed in a plurality of reaction vessels, which may be provided as wells in a storage device according to the present disclosure. For example, various starting compounds may be prepared employing one or more of solid-phase synthesis, recorded random mix methodologies and recorded reaction split techniques that permit a given constituent to traceably undergo a plurality of permutations and/or combinations of reaction conditions. The resulting products comprise a library that can be screened followed by iterative selection and synthesis procedures, such as a synthetic combinatorial library of peptides (see e.g., PCT/US91/08694 and PCT/US91/04666) or other compositions that may include small molecules as provided herein (see e.g., PCT/US94/08542, EP 0774464, U.S. Pat. No. 5,798,035, U.S. Pat. No. 5,789,172, U.S. Pat. No. 5,751,629).

### System for Storing, Tracking, and Retrieving Data Associated

### With Biological Materials

The foregoing storage device in the various embodiments described above can be combined with other technologies to provide for integration of sample storage and sample management for life science applications. This embodiment of the invention enables the integration of biological sample storage, location, tracking, processing, and sample data management. Data regarding samples can be associated with the location of the samples through direct physical association of the data with the sample storage devices. The stored information can be updated with additional data that originates from inventory and tracking of samples in combination with multi-step biological research protocols, production processes, screening, bioassays, patient histories, clinical trial data, and other sources of developed information. The data associated with the sample can be transmitted and shared through a secure hierarchical software and networking architecture that enables interfacing of multi-user, multi-site environments.

Ideally, information about a sample is integrated with the sample storage device by an associated electronic interface, preferably a wireless interface, such as a radio frequency identification (RFID) transponder. While barcodes have been used in the past to identify samples, this technology has limitations that make it unsuitable for use in the present invention. These limitations include the required line-of-sight access to the bar code for transfer of information, limited information capacity, and interference through environmental factors such as dust, moisture, and the like. Radio frequency identification technology overcomes these disadvantages.

Remote communication utilizing wireless equipment typically relies on radio frequency (RF) technology, which is employed in many industries. One application of RF technology is in locating, identifying, and tracking objects, such as animals, inventory, and vehicles. Examples of publications disclosing RF identification tag systems include the disclosures of U.S. Pat. Nos. 6,696,028; 6,380,858; and 5,315,505.

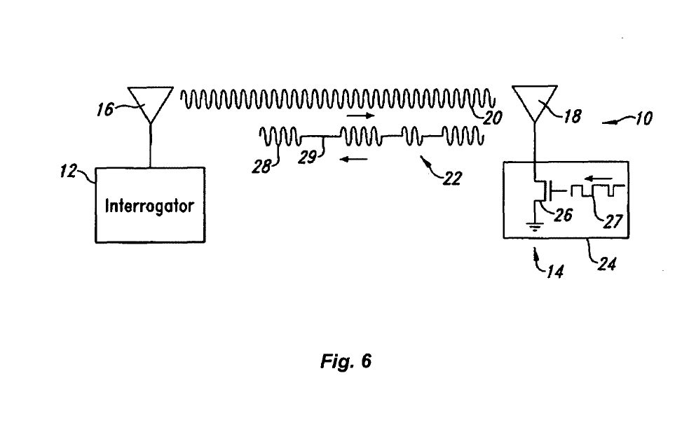

RF identification (RFID) tag systems have been developed that facilitate monitoring of remote objects. As shown in FIG. 6, a basic RFID system 10 includes two components: an interrogator or reader 12, and a transponder (commonly called an RF tag) 14. The interrogator 12 and RF tag 14 include respective antennas 16, 18. In operation, the interrogator 12 transmits through its antenna 16 a radio frequency interrogation signal 20 to the antenna 18 of the RF tag 14. In response to receiving the interrogation signal 20, the RF tag 14 produces an amplitude-modulated response signal 22 that is transmitted back to the interrogator 12 through the tag antenna 18 by a process known as backscatter.

The conventional RF tag 14 includes an amplitude modulator 24 with a switch 26, such as a MOS transistor, connected between the tag antenna 18 and ground. When the RF tag 14 is activated by the interrogation signal 20, a driver (not shown) creates a modulating on/off signal 27 based on an information code, typically an identification code, stored in a non-volatile memory (not shown) of the RF tag 14. The modulating signal 27 is applied to a control terminal of the switch 26, which causes the switch 26 to alternately open and close. When the switch 26 is open, the tag antenna 18 reflects a portion of the interrogation signal 20 back to the interrogator 12 as a portion 28 of the response signal 22. When the switch 26 is closed, the interrogation signal 20 travels through the switch 26 to ground, without being reflected, thereby creating a null portion 29 of the response signal 22. In other words, the interrogation signal 20 is amplitude-modulated to produce the response signal 22 by alternately reflecting and absorbing the interrogation signal 20 according to the modulating signal 27, which is characteristic of the stored information code. The RF tag 14 could also be modified so that the interrogation signal is reflected when the switch 26 is closed and absorbed when the switch 26 is open. Upon receiving the response signal 22, the interrogator 12 demodulates the response signal 22 to decode the information code represented by the response signal. The conventional RFID systems thus operate on a single frequency oscillator in which the RF tag 14 modulates a RF carrier frequency to provide an indication to the interrogator 12 that the RF tag 14 is present.

The substantial advantage of RFID systems is the non-contact, non-line-of-sight capability of the technology. The interrogator 12 emits the interrogation signal 20 with a range from one inch to one hundred feet or more, depending upon its power output and the radio frequency used. Tags can be read through a variety of substances such as odor, fog, ice, paint, dirt, and other visually and environmentally challenging conditions where bar codes or other optically-read technologies would be useless. RF tags can also be read at remarkable speeds, in most cases responding in less than one hundred milliseconds.

A typical RF tag system 10 often contains a number of RF tags 14 and the interrogator 12. RF tags are divided into three main categories. These categories are beam-powered passive tags, battery-powered semi-passive tags, and active tags. Each operates in fundamentally different ways.

The beam-powered RF tag is often referred to as a passive device because it derives the energy needed for its operation from the interrogation signal beamed at it. The tag rectifies the field and changes the reflective characteristics of the tag itself, creating a change in reflectivity that is seen at the interrogator. A battery-powered semi-passive RF tag operates in a similar fashion, modulating its RF cross-section in order to reflect a delta to the interrogator to develop a communication link. Here, the battery is the source of the tag's operational power. Finally, in the active RF tag, a transmitter is used to create its own radio frequency energy powered by the battery.

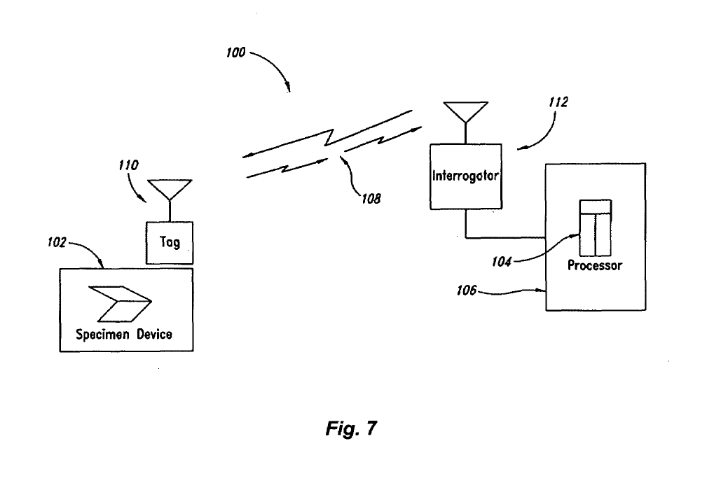

In a preferred embodiment of the present invention, the system consists of three parts, a consumable hardware device, inventory and management software, and the RFID interface between the hardware device and the software. Referring to FIG. 7, shown therein is a system 100 formed in accordance with one embodiment of the invention to include the storage device 102 described above, the inventory and management software component 104, preferably implemented in a computer system 106, and the radio frequency identification interface 108 coupling the storage device 102 and the software 106. Preferably, the RFID interface 108 includes a transponder 100 associated with the storage device 102 and an interrogator 112, which is coupled to the computer-implemented system 106.

In this embodiment, the transponder 110 is associated with the sample storage device 102, such as by affixing the transponder 110 to an exterior surface of the storage device 102. However, it is to be understood that the transponder 110 can be affixed to or associated with a tube, a plate, a rack, or even a room in which the storage device 102 is maintained. While it is preferred that a single transponder 110 be associated with a single storage device 102, it is possible that each particular sample stored in the storage device 102 can have a transponder 110 associated with it.

Association can be achieved either during production of the storage device 102 such that the transponder 110 is embedded in the storage device 102 or after the storage device 102 has been produced, such as through adhesive affixation to the storage device 102. Inasmuch as magnetism is the preferred connecting mechanism used in the sample storage device 102 in its various embodiments, it will be understood by one of ordinary skill in this technology that appropriate shielding may be needed to prevent unintentional altering of information stored in the transponder 110 and to prevent interference with radio frequency communications between the transponder 110 and the interrogator 112.

The transponder 110 can be preprogrammed with data about the storage device 102 and the samples stored in the storage device 102, including ownership information, location information, analysis information, production processes, clinical trial conduct, synthesis processes, sample collections, and other information known to those skilled in the art that would be of value in managing samples. In addition to preprogramming such data, the transponder 110 can be configured to permit modification and updating of the data within its memory. In addition, the transponder 110 will contain security architecture that defines precise access conditions per type of data to thereby restrict reading, writing, and updating. For example, the RFID interface 108 components can be configured to receive control signals from and to respond to a particular computer-implemented data processing system, such as the software application described herein below. In addition, data written to the transponder 110 can be encrypted for authentication and security purposes.

The use of RFID transponders or chips offers the benefit of a wide temperature range (−25° C. to +85° C.) without the loss of functionality. In addition, the transponders 110 can be utilized to control remote devices, such as a signaling light or generator of audible tones for alerting and locating the object associated with the transponder 110. Storage of information in the transponder 110 also provides an additional backup should data in the computer-implemented system 106 be damaged or lost.

The interrogator 112 is a conventional radio frequency identification reader that is coupled to the computer-implemented system 106. Command and control signals are generated by the system 106 to initiate interrogation of one or more transponders 110 and to receive a response therefrom that is processed by the software 104 in the computer-implemented system 106. In one configuration, the transponders 110 can be reprogrammed via communications from the interrogator 112 to replace or update data stored therein.

In one implementation, one or more interrogators 112 are positioned within a facility at a sufficient range to communicate via radio frequency signals, such as microwave signals, with the transponders 110. Multiple interrogators 112 can be used for multiple classes of transponders 110 or with individual transponders 110. Alternatively, one interrogator utilizing known technology can communicate with multiple transponders 110 on multiple frequencies in serial fashion or concurrently. In applications where a sample storage device 102 or individual samples are processed, multiple interrogators positioned at various locations within a structure or along a path of travel, such as a conveyor system or a shipping system, such as freight lines, trains, and the like, can be used to track the location and the status of the sample. This includes checking environmental factors, such as temperature, humidity, pressure, and the like in which the specimen or storage device 102 is located.

Thus, the RFID interface 108 can be expanded to monitor and process data related to the movement and analysis of a sample or storage device 102 located in a laboratory, manipulated by laboratory robots, and the like such as during biological production processes or the execution of experimental steps. This also aids in quality control and in processing biological samples through automated or semi-automated research protocols.

As mentioned above, sample storage and tracking are facilitated by locating a sample through the use of an RF interface between the RF transponder on the sample storage device and the computer-implemented system described herein, which is achieved through the tagging and monitoring of the storage location, such as a storage rack, a storage room, a refrigerator, a lab bench, a desk, or a bookshelf.

In order to trace a particular storage device 102 or sample, the transponder 110 is configured to activate a remote device, such as a blinking light located on the storage device, an audible device associated with the storage device, or a color change of the storage device that can be recognized by a person or by an automated system, to enable fast retrieval of the sample. In addition, the transponder 110 is configured to activate a remote alarm when an environmental condition has exceeded a predetermined environmental range, including but not limited to temperature, pressure, and humidity. In one embodiment, the transponder 110 is a passive device that is activated by the interrogation signal, from which it draws operating power. When the transponder 110 is used to activate a remote device or to increase the range of communication, the transponder can be semi-active as described above. Alternatively, an active transponder can be used when large amounts of data are to be read from or written to the transponder 110 or increased range as desired. Range is also affected by frequency, as is known in the art, and one of ordinary skill would select the appropriate frequency range in accordance with the environment, and the functional objectives. For example, certain specimens may be sensitive to particular frequencies of radio signals, and such frequencies would need to be avoided or the specimen appropriately shielded when designing the system 100.

The inventory and management software 104 is tailored for use with wireless communication systems and the processing of data associated with the life sciences. It consists of a customized user interface and a set of predefined database tables in one embodiment. A user can enter sample-associated data or import information from outside sources. Predefined tables are provided in the database to facilitate setup of the system, but a user can have the option to customize fields within the tables. The relational database can include tables for DNA sample, clones, oligonucleotides, PCR fragments, cDNA, chemical compounds, proteins, metabolites, lipids, cellular fractions, biological samples from different organisms such as viruses, bacteria, or multi-cellular organisms, patient samples such as blood, urine, and buccal swabs. Detailed sample information and sample-associated data is programmed into the tables. Sample information can for example include sample source, clone name, gene insert name, insert size, insert sequence, modifications, vector name, vector size, antibiotic selection, induction, terminator, cloning sight, 5′-tag, 3′-tag, purification tag, oligonucleotide name, purification, quality control, forward primer, reverse primer, Tₘ value, and size selection. Clinical patient information can be, for example, age, gender, location, ethnic group, body mass index, family history, medication, data of onset of symptoms, duration of disease, and medical tests. Sample-associated data can consist of research data from various sources, such as, for example, sequence information from a DNA sequencer, transcriptional profiling information from microarray chips, protein data from Western blotting or in-situ hybridization, bioassay data for drug discovery, high through-put drug screening data, chemical library synthesis data, and the like. Data can be supplied in the form of text, numbers, tables, or images.

The software can also link to other data sources and integrate information from public domains, such as GenBank, SwissProt, and other similar domains or proprietary sources. Ideally the software is able to interface with robotics equipment to track the sample within a process, and tracking of the process can be displayed as an accumulative sample history for storage within the sample device as well as the database, such as storage in an RFID transponder 110.

The software is designed to create an informatics infrastructure where a single user generates their data and information set, which is initially stored at a local workstation in a local database format. However, the software is capable of linking multiple users in a hierarchical environment. The information accumulated by a single user can best be up-loaded to a centralized database system on a server. The interaction of the network environment can also be a web browser interface. The multi-user environment can be expanded to multiple-site environments, and software and databases can be located on a personal computer, on a server within an intranet or on the internet such as an e-commerce site. Access control and log control systems are also provided in the software.

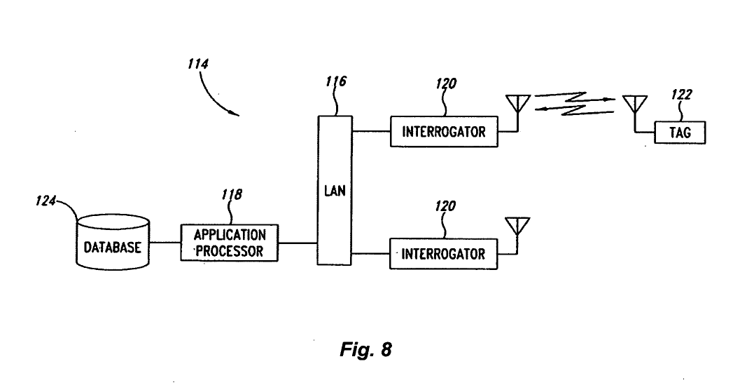

Shown in FIG. 8 is a computer-implemented system architecture 114 for utilizing a local area network 116 to interface an application processor 118 with one or more interrogators 120 that communicate with one or more remote RFID tags 122. The application processor 118 is coupled to a database 124 It is to be understood that the local area network can instead be a global network, such as the Internet, in which case web-based applications would be utilized.

Ideally, in one embodiment the inventory and management software 104 has three components, a front end software component, a middleware component, and a back end software component.

It is envisioned that the front end software is utilized to create a “user interface.” This can be, for example, a web browser, Microsoft Excel or a similar grid component. The web browser software would be used for a web-based system 100, whereas the Microsoft Excel software would be used for a desktop system. The web-based option provides for multiple users, networking, and can be expanded to accommodate thousands of users. The desktop option is sufficient for a single user who does not anticipate sharing of data and sample information via a network.

The middleware can include Microsoft Excel macros or grid components developed for use as a desktop option or custom software created by programming language suitable for use with web-based systems, such as PHP. The middleware is configured as a collection of programs that is capable of receiving user inputs and queries and returning database information to the user via known output, such as printer, display, or audible output.

The back end software is preferably Microsoft Access, which is proprietary database software offered by Microsoft Corporation and hosted by Microsoft Excel. This particular program provides sufficient database capacity to support up to 50,000 records, and to a maximum of 100,000 records with increasing levels of performance degradation. Another option is MySQL, which is a freeware database software developed collaboratively and available at no charge that runs on all major servers, including those based on Windows and Linux platforms. This database is capable of handling millions of records, and would be suitable for the large institutional user, such as governmental agencies, universities, and multinational entities.

The software 104 is configured to provide control signals to the RFID interface 108 and to receive data and information from the interface 108. In addition, when information is supplied to a transponder, the software 104 is configured to initiate writing of the data through the interrogator 112 to the transponder 110 using methods and equipment known in the art and which is readily commercially available.

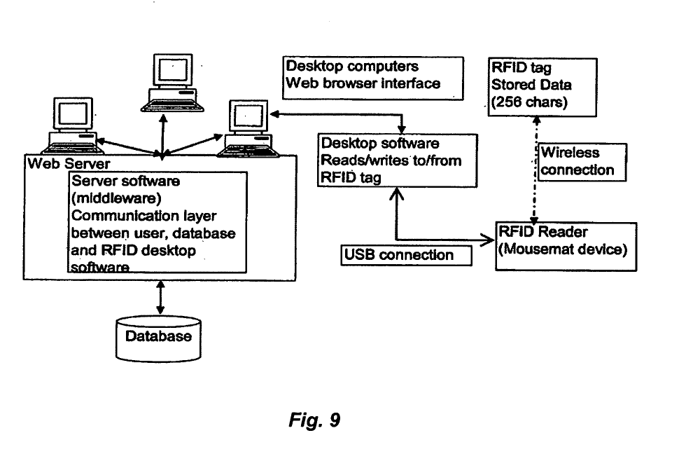

FIG. 9 illustrates another system architecture 128 in which a database 130 is linked to a plurality of desktop computers 132 via a web server 134. Resident on the server 134 is software that provides a communication layer between the user, the database 130, and desktop software 136 resident on the desktop computers 132. With a web browser interface 138, a user can connect to the RFID reader 142 through a standard USB connection 140. The user can then control read and write operations of the RFID reader 142 and the remote RFID tag 144 using the wireless connection 146 provided by the radio frequency communications.

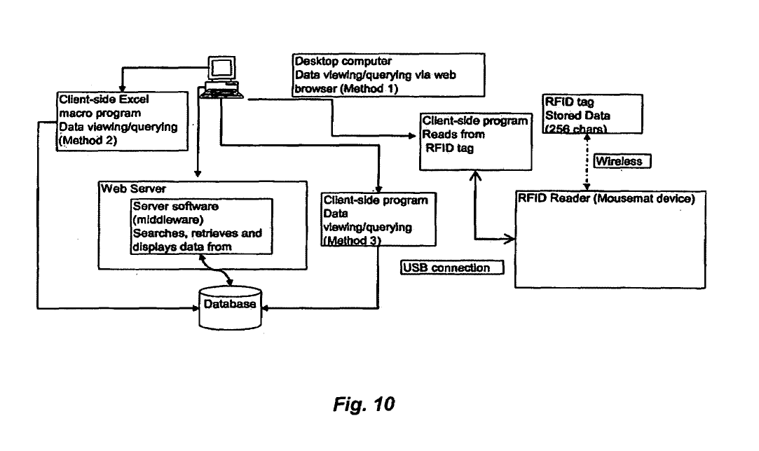

Referring next to FIG. 10, shown therein is a further embodiment of the invention utilizing a 3-tier architecture 148 having a desktop computer 150 with a front-end web browser 158 linked to a backend database 154 via web server middleware 156 on a web server 152. The middleware search, retrieval, and display ability to a user. More particularly, the business logic is contained in the middleware program 156 on the web server 152. In addition, there is (optionally) an RFID reader 160 coupled via a USB connection 162 to the client-side program 164 on the desktop computer 150. The client-side application, which reads and writes to the RFID tag 166 via the reader 160, is launched from the web browser 158.

In an alternative 2-tier arrangement of this architecture 148, there is an Excel front-end program on the desktop computer 150 that communicates directly with the database 154 at the back end. The business logic here is embodied in the Excel macro program. This method is particularly efficient for loading data (e.g., 96 rows of data corresponding to each well in a plate) into a database to take advantage of the Excel functions, such as copying, dragging down, etc.

In a further alternative 2-tier arrangement of the architecture 148, a stand-alone client application 170 at the front end communicates directly with the database 154 at the back end. The business logic is contained within the stand-alone client application, and a module for reading from and writing to the RFID tag 166 may also be contained within this application 170. Here the advantage is that the application is compiled (the source code is not visible) and does not require third-party software (Excel, web-server). The drawback is that it is not as network compatible as the 3-tier architecture described above.

The following Examples are presented by way of illustration and not limitation.

### EXAMPLES

### Example 1

### Storage of Blood

This Example describes the preparation and characterization of a liquid-storable biological sample. In this and the following Examples, standard cell and molecular biology techniques were employed, essentially according to known methodologies (e.g., Sambrook, J., Fritsch, E. F. and Maniatis, T. (1989) Molecular Cloning: A Laboratory Manual, Cold Spring Harbor Laboratory Press, New York; Current Protocols, Nucleic Acid Chemistry, Molecular Biology, Wiley and Sons, 2003; Current Protocols, Protein Sciences, Cell Biology, Wiley and Sons, 2003). All reagents in this and the following Examples were from Sigma-Aldrich (St. Louis, Mo.) unless otherwise specified.

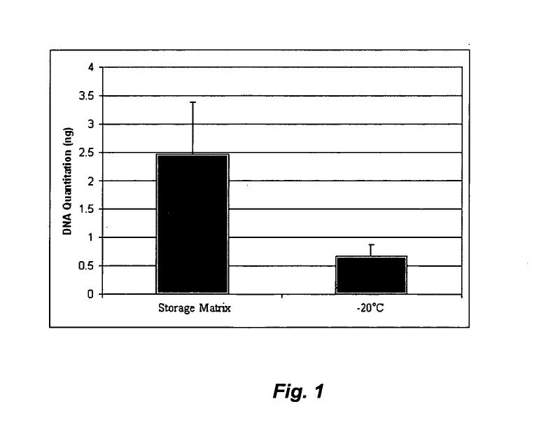

For room temperature storage of blood, 10 mM Tris pH 8.0 was used for the preparation of a 1% polyvinyl alcohol (PVA, Sigma-Aldrich no. P8136) basic liquid storage matrix. The concentration of the polymer was tested in a range of 0.1% to 10% (w/v). The pH of the matrix was tested in a range of pH 5 to 8. For convenient detection of biological sample, phenol red was added to the liquid matrix at 0.002% (w/v). A sample of 20 μl whole blood was mixed with 50 μl of 1% PVA basic liquid storage matrix, sealed and stored in a polypropylene 96-well plate at ambient (room) temperature. An equal volume of whole blood was stored at −20° C. in a sealed polypropylene 96-well plate without matrix. After 4 months, genomic DNA was extracted from the frozen and room temperature stored samples using phenol/chloroform. A 5 μl aliquot of extracted DNA was used for quantitative PCR (QPCR) in triplicate. QPCR was conducted using SYBR green (Applied Biosystems, Inc., Foster City, Calif.; “ABI”) technology on the ABI 7300 sequence detection system (ABI) and primers for human beta-actin were designed with Primer Express 3.0. The sequence for the human beta-actin forward primer was 5′ ACCGAGCGCGGCTACAG [SEQ ID NO: 1] and human beta-actin reverse primer was 5′CTTAATGTCACGCACGATTTCC [SEQ ID NO: 2]. Each sample contained 5 μl template, 6.5 μl Power SYBR green PCR master mix (ABI) and 0.5 μl of each primer (10 μM final concentration) with a total final volume of 25 μl. The cycling parameters used were an initial denaturation at 95° C. for 10 min, followed by 40 cycles of 95° C. for 15 s and 60° C. for 1 min. Results are shown in FIG. 1. Genomic DNA derived from blood stored at room temperature in liquid storage matrix retained significant amounts of intact genomic DNA as assayed by QPCR, as compared to blood stored frozen without storage matrix.

### Example 2

### Storage of RNA

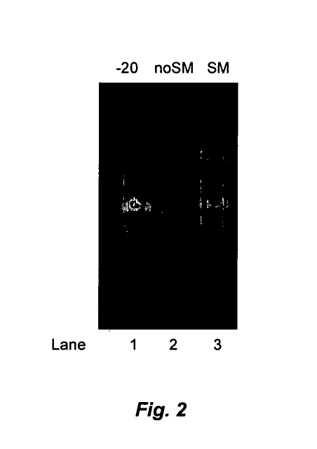

A 1 μg sample of ssRNA ladder (New England Biolabs, Inc., Beverly, Mass.; “NEB”) was suspended in 10 μl 1% PVA basic liquid storage matrix (prepared as described above in Example 1) and an equal amount of a control sample was suspended in water and both preparations were stored on the laboratory bench top at ambient (room) temperature. An additional sample in basic liquid storage matrix was stored frozen at −20° C. After 6 days, samples were supplemented with 10 μl RNA gel loading dye (NEB) and electrophoretically separated on a 0.8% agarose gel, which was then stained with ethidium bromide to visualize the RNA. Results are shown in FIG. 2. RNA stored in water was significantly degraded as compared to samples in liquid storage matrix kept at either −20° C. or at room temperature.

### Example 3

### Storage of Plasmid DNA

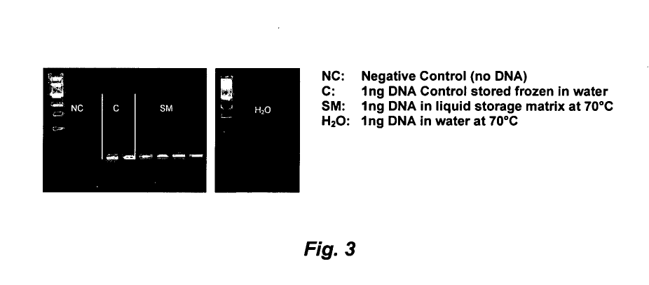

A 1 ng PDNA (pUC19) sample (NEB) was resuspended in 1% PVA basic liquid storage matrix (prepared as described above in Example 1), or in water. The samples were placed in a 70° C. oven for 3 days. A control sample was stored in water at −20° C. For PCR analysis each reaction contained 2.5 U Taq DNA polymerase (New England Biolabs, Inc.), 3 μl 10× reaction buffer (NEB), 0.5 μl dNTPs (10 μM each nucleotide), pUC19 forward primer (5′-ACCGCACAGATGCGTAAGGAG) [SEQ ID NO: 3] and pUC19 reverse primer (5′-TTCATTAATGCAGCTGGCACG) [SEQ ID NO: 4] each at a final concentration of 0.2 μM in a final volume of 30 μl. Cycling parameters were an initial denaturation at 94° C. for 5 min followed by 30 cycles of 94° C. for 15 sec, 55° C. for 30 sec and 72° C. for 30 sec. PCR reactions (10 μl) were analyzed by 0.8% agarose gel electrophoresis followed by ethidium bromide staining. Results are shown in FIG. 3. The integrity of plasmid DNA stored in water at room temperature was apparently compromised, as the material could not be amplified. The integrity of plasmid DNA maintained in liquid storage matrix, however, was sufficient such that the stored material could be amplified without significant loss of amplification efficiency as compared to control samples.

### Example 4

### Storage of Enzymes

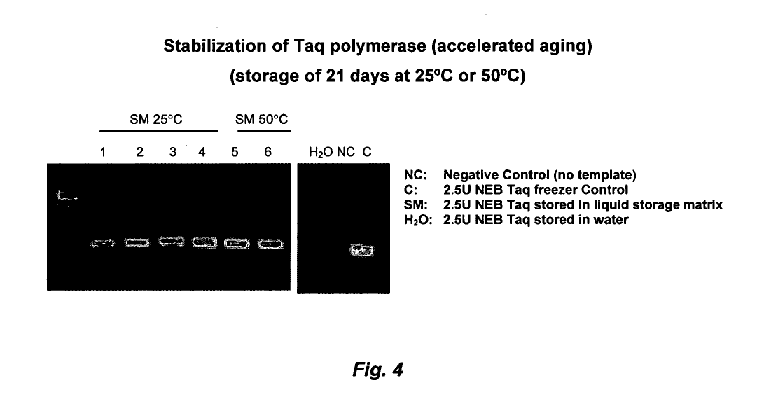

Samples of 2.5 U Taq Polymerase (NEB) were added to 10 μl 1% PVA basic liquid storage matrix (prepared as described above in Example 1), or 10 μl water, and stored at 25° C. or 50° C. for 21 days (accelerated aging conditions). An additional sample of Taq polymerase was stored at −20° C. as a positive control. For PCR analysis, each reaction contained 50 ng pUC19 plasmid DNA, 3 μl 10× reaction buffer (NEB), 0.5 μl dNTPs (10 μM each nucleotide), pUC19 forward primer (5′-ACCGCACAGATGCGTAAGGAG) [SEQ ID NO: 3] and pUC19 reverse primer (5′-TTCATTAATGCAGCTGGCACG) [SEQ ID NO: 4] each at a final concentration of 0.2 μM in a final volume of 30 μl. Cycling parameters were an initial denaturation at 94° C. for 5 min followed by 30 cycles of 94° C. for 15 sec, 55° C. for 30 sec and 72° C. for 30 sec. 10 μl of each PCR reaction was analyzed by 0.8% agarose gel electrophoresis followed by staining of the gel with ethidium bromide. Results are presented in FIG. 4. Taq polymerase stored in liquid storage matrix at either room temperature (25° C.) or 50° C. was capable of enzymatic activity in PCR reactions, while enzyme stored in water was unable to amplify nucleic acid, indicating loss of enzymatic function and integrity.

### Example 5

### Storage of Bacterial Cells

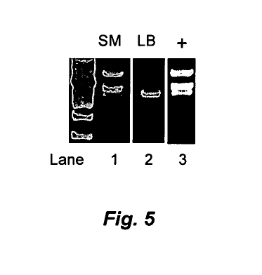

A liquid overnight culture of Stb12 E. coli (Invitrogen, Carlsbad, Calif.) harboring the PFIV-C plasmid (13 kb) was used. A 5 μl sample of the culture was mixed with 20 μl 1% PVA basic liquid storage matrix (prepared as described above in Example 1) or liquid Luria Broth (LB) in tubes and stored at room temperature for 2 months. Samples were then used to inoculate 3 ml of LB/ampicillin (10 mg/ml) and grown overnight on a shaker at 37° C. for 18 h. Plasmid DNA was extracted using the alkaline lysis method (Sambrook, J., Fritsch, E. F. and Maniatis, T. (1989) Molecular Cloning: A Laboratory Manual, Cold Spring Harbor Laboratory Press, New York). The purified plasmid DNA was then digested with EcoRI (NEB) and run alongside control plasmid DNA on a 0.8% agarose gel, which was then stained with ethidium bromide. Results are shown in FIG. 5. Plasmid DNA of the expected size was detected from bacteria derived from glycerol stocks (positive control) or from cells maintained in liquid matrix storage at room temperature. A band of the correct size was absent in samples derived from cells stored in LB at room temperature, indicating loss of plasmid when bacteria were maintained under such conditions.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

1. A liquid-storable biological sample, comprising:
(a) a biological sample;
(b) a liquid matrix that comprises a matrix material dissolved or dissociated in a biocompatible solvent; and
(c) at least one stabilizer,
wherein (a), (b) and (c) are in fluid contact with one another for at least one day without refrigeration, and
wherein substantially all biological activity of the liquid-storable biological sample is recoverable following storage without refrigeration for a time period of at least one day.

2. The liquid-storable biological sample of claim 1 which comprises at least two stabilizers.

3. The liquid-storable biological sample of claim 1 wherein the at least one stabilizer comprises a trehalase inhibitor.

4. The liquid-storable biological sample of claim 1 wherein the matrix material comprises polyvinyl alcohol.

5. The liquid-storable biological sample of claim 1 wherein the at least one stabilizer comprises a glycosidase inhibitor that is selected from the group consisting of:
(i) a trehalase inhibitor,
(ii) a chitinase inhibitor,
(iii) an α-glucosidase inhibitor,
(iv) a glycogen phosphorylase inhibitor,
(vi) a neuraminidase inhibitor,
(vi) a ceramide glucosyltransferase inhibitor, and
(vii) a lysosomal glycosidase inhibitor.

6. The liquid-storable biological sample according to either claim 3 or claim 5 wherein the trehalase inhibitor is selected from the group consisting of suidatrestin, validamycin A, validoxylamine A, MDL 26537, trehazolin, salbostatin and casuarine-6-O-α-D-glucopyranoside.

7. The liquid-storable biological sample according to claim 1 wherein the matrix material is dissolved in the biocompatible solvent.

8. The liquid-storable biological sample according to claim 1 wherein at least one stabilizer comprises an inhibitor that is a biological inhibitor or a biochemical inhibitor.

9. The liquid-storable biological sample of claim 5 wherein the matrix material comprises polyvinyl alcohol.

10. The liquid-storable biological sample according to claim 1 wherein the liquid matrix comprises a solution that comprises from about 0.1% to about 10% weight-to-volume polyvinyl alcohol.

11. The liquid-storable biological sample of claim 10 wherein the liquid matrix comprises a solution that comprises from about 0.5% to about 5% weight-to-volume polyvinyl alcohol.

12. The liquid-storable biological sample of claim 10 wherein the liquid matrix comprises a solution that comprises from about 1% to about 5% weight-to-volume polyvinyl alcohol.

13. The liquid-storable biological sample of claim 10 wherein the liquid matrix comprises a solution comprises from about 0.5% to about 1.5% weight-to-volume polyvinyl alcohol.

14. The liquid-storable biological sample of claim 10 wherein the liquid matrix comprises a solution that is selected from the group consisting of:
(i) a solution that comprises about 1% weight-to-volume polyvinyl alcohol,
(ii) a solution that comprises about 3% weight-to-volume polyvinyl alcohol,
(iii) a solution that comprises about 5% weight-to-volume polyvinyl alcohol,
(iv) a solution that comprises about 1% weight-to-volume polyvinyl alcohol and about 5% weight-to-volume trehalose,
(v) a solution that comprises about 1% weight-to-volume polyvinyl alcohol and about 5% weight-to-volume validamycin, and
(vi) a solution that comprises about 1% weight-to-volume polyvinyl alcohol, about 5% weight-to-volume trehalose and about 5% weight-to-volume validamycin.

15. The liquid-storable biological sample of claim 10 wherein the liquid matrix comprises a solution that is selected from the group consisting of:
(i) a solution that comprises from about 1% weight-to-volume to about 5% weight-to-volume polyvinyl alcohol and about 5% weight-to-volume of a trehalase inhibitor,
(ii) a solution that comprises about 1% weight-to-volume polyvinyl alcohol and about 1% to about 10% weight-to-volume of a trehalase inhibitor, and
(iii) a solution that comprises about 1% weight-to-volume polyvinyl alcohol, about 5% weight-to-volume trehalose and about 5% weight-to-volume of a trehalase inhibitor.

16. The liquid-storable biological sample according to claim 14 or claim 15 wherein the trehalase inhibitor is selected from the group consisting of suidatrestin, validamycin A, validoxylamine A, MDL 26537, trehazolin, salbostatin and casuarine-6-O-α-D-glucopyranoside.

17. The liquid-storable biological sample of claim 1 wherein the matrix material comprises at least one material selected from the group consisting of polyethylene glycol, agarose, poly-N-vinylacetamide, polyvinyl alcohol, carboxymethyl cellulose, 2-hydroxyethyl cellulose, poly(2-ethyl-2-oxazoline), poly(vinyl-pyrrolidone)sui, poly(4-vinylpyridine), polyphenylene oxide, crosslinked acrylamide, polymethacrylate, carbon nanotubes, polylactide, lactide/glycolide copolymer, hydroxymethacrylate copolymer, calcium pectinate, hydroxypropyl methylcellulose acetate succinate, heparin sulfate proteoglycan, hyaluronic acid, glucuronic acid, thrombospondin-1 N-terminal heparin-binding domain, fibronectin, a peptide/water-soluble polymeric modifier conjugate and collagen.

18. (canceled)

19. The liquid-storable biological sample of claim 1 wherein the biological sample comprises at least one of
(i) an isolated biomolecule that is selected from the group consisting of DNA, RNA, a protein, a polypeptide, a lipid, a carbohydrate, a glycoconjugate, an oligosaccharide, and a polysaccharide,
(ii) a biological material that is selected from the group consisting of a mammalian cell, a non-mammalian cell, a plant cell, an animal cell, a bacterium, a microorganism, a yeast cell, a virus, a vaccine, blood, urine, a biological fluid, an environmental sample, and a buccal swab, and
(iii) a bioactive small molecule.

20. A liquid-storable biological sample, comprising:
(a) a biological sample;
(b) a liquid matrix that comprises polyvinyl alcohol dissolved in a biocompatible solvent; and
(c) at least one stabilizer which comprises validamycin,
wherein (a), (b) and (c) are in fluid contact with one another for at least one day without refrigeration, and wherein substantially all biological activity of the liquid-storable biological sample is recoverable following storage without refrigeration for a time period of at least one day.

21. The liquid-storable biological sample according to claim 1 or claim 20, further comprising a buffer that is capable of maintaining a desired pH.

22. The liquid-storable biological sample of claim 21 wherein the buffer comprises a compound that is selected from the group consisting of Tris, citrate, acetate, phosphate, borate, HEPES, MES, MOPS, PIPES, carbonate and bicarbonate.

23. The liquid-storable biological sample of claim 8 wherein the biological inhibitor or biochemical inhibitor is selected from the group consisting of validamycin A, TL-3, sodium orthovanadate, sodium fluoride, N-α-tosyl-Phe-chloromethylketone, N-α-tosyl-Lys-chloromethylketone, aprotinin, phenylmethylsulfonyl fluoride and diisopropylfluoro-phosphate.

24. The liquid-storable biological sample of claim 8 wherein the biological inhibitor or biochemical inhibitor is selected from the group consisting of a kinase inhibitor, a phosphatase inhibitor, a caspase inhibitor, a granzyme inhibitor, a cell adhesion inhibitor, a cell division inhibitor, a cell cycle inhibitor, a lipid signaling inhibitor and a protease inhibitor.

25. The liquid-storable biological sample of claim 8 wherein the biological inhibitor or biochemical inhibitor is selected from the group consisting of a reducing agent, an alkylating agent and an antimicrobial agent.

26. The liquid-storable biological sample according to claim 1 or claim 20, which comprises at least one detectable indicator.

27. The liquid-storable biological sample of claim 26 wherein the detectable indicator comprises a calorimetric indicator.

28. The liquid-storable biological sample of claim 26 wherein the detectable indicator comprises one or a plurality of gas chromatography/mass spectrometry (GCMS) tag compounds.

29. The liquid-storable biological sample of claim 26 wherein the detectable indicator is selected from the group consisting of a fluorescent indicator, a luminescent indicator, a phosphorescent indicator, a radiometric indicator, a dye, an enzyme, a substrate of an enzyme, an energy transfer molecule, and an affinity label.

30. The liquid-storable biological sample of claim 26 wherein the detectable indicator is capable of detectably indicating presence of at least one of an amine, an alcohol, an aldehyde, a thiol, a sulfide, a nitrite, avidin, biotin, an immunoglobulin, an oligosaccharide, a nucleic acid, a polypeptide, an enzyme, a cytoskeletal protein, a reactive oxygen species, a metal ion, pH, Na⁺, K⁺, Cl⁻, a cyanide, a phosphate and selenium.

31. The liquid-storable biological sample of claim 26 wherein the detectable indicator is selected from the group consisting of phenol red, ethidium bromide, a DNA polymerase, a restriction endonuclease, cobalt chloride, Reichardt's dye and a fluorogenic protease substrate.

32. The liquid-storable biological sample of claim 1 or claim 20 wherein substantially all biological activity of the liquid-storable biological sample is recoverable following storage without refrigeration for a time period that is selected from the group consisting of (i) at least one week, (ii) at least one month, (iii) at least six months, (iv) at least nine months, (v) at least twelve months, (vi) at least eighteen months and (vii) at least twenty-four months.

33. The liquid-storable biological sample according to claim 1,
wherein:
(I) the matrix material does not covalently self-assemble and has the structure:
—[—X—]ₙ—
wherein X is —CH₃, —CH₂—, —CH₂CH(OH)—, substituted —CH₂CH(OH)—, —CH₂CH(COOH)—, substituted —CH₂CH(COOH)—, —CH═CH₂, —CH═CH—, C₁-C₂₄ alkyl or substituted alkyl, C₂₋₂₄ alkenyl or substituted alkenyl, polyoxyethylene, polyoxypropylene, or a random or block copolymer thereof;
and wherein n is an integer having a value of about 1-100, 101-500, 501-1000, 1001-1500, or 1501-3000;
and wherein
(II) the stabilizer is not covalently linked to the matrix material and comprises trehalose, a trehalase inhibitor, or a compound that is selected from the group consisting of D-(+)-raffinose, β-gentiobiose, ectoine, D-(+)-raffinose pentahydrate, myo-inositol, hydroxyectoine, magnesium D-gluconate, 2-keto-D-gluconic acid hemicalcium salt hydrate, D(+)-melezitose, calcium lactobionate monohydrate, β-lactose, turamose, and D-maltose.

34. The liquid-storable biological sample of claim 33 wherein the matrix material is capable of non-covalent association with at least one stabilizer.

35. The liquid-storable biological sample of claim 33 wherein the matrix material is capable of non-covalent association with at least one of a nucleic acid molecule and a polypeptide.

36.-53. (canceled)

54. A liquid-storable biological sample, comprising:
(a) a biological sample;
(b) a liquid matrix that comprises a matrix material dissolved or dissociated in a biocompatible solvent;
(c) at least one stabilizer; and
(d) an activity buffer,
wherein (a), (b), (c) and (d) are in fluid contact with one another for at least one day without refrigeration, and
wherein substantially all biological activity of the liquid-storable biological sample is recoverable following storage without refrigeration for a time period of at least one day.

55. The liquid-storable biological sample of claim 54 wherein the activity buffer comprises a composition that is selected from the group consisting of a pH buffer, a free radical trapping agent, and a pathogen-neutralizing agent.

56.-68. (canceled)

69. The liquid-storable biological sample according to any one of claims 1, 20 and 54 that is formulated to be isotonic, hypertonic or hypotonic.

70.-72. (canceled)
